(19) United States
(12) Patent Application Publication
(10) Pub. No.: US 2019/0117922 A1
(43) Pub. Date: Apr. 25, 2019

(54) FIXATION DEVICE

(71) Applicant: TELEFLEX LIFE SCIENCES UNLIMITED COMPANY, Hamilton (IE)

(72) Inventors: Sylwia Poulsen, Singapore (SG); Teck Poh, Singapore (SG)

(21) Appl. No.: 16/090,137

(22) PCT Filed: Mar. 31, 2017
(86) PCT No.: PCT/EP2017/057774

§ 371 Date: Sep. 28, 2018

(30) Foreign Application Priority Data
Mar. 31, 2016 (GB) 1605505.5

(51) Int. Cl. A61M 16/04; A61M 16/06

(52) U.S. Cl. CPC A61M 16/0409; A61M 16/0434; A61M 16/0497; A61M 16/0683

(57) ABSTRACT

A fixation device (8) for use with a laryngeal mask airway device (10), the fixation device (8) comprising releasable locking means (12) for releasably securing the fixation device to the laryngeal mask airway device.

The present invention relates to a fixation device, and more particularly to a fixation device that may be used with a laryngeal mask airway device.

Artificial airway devices such as the laryngeal mask airway device are well known devices useful for establishing airways in unconscious patients. In its most basic form a laryngeal mask airway device consists of an airway tube and a mask carried at one end of the airway tube, the mask having a peripheral formation often known as a “cuff” which is capable of conforming to and of fitting within, the actual and potential space behind the larynx of the patient so as to form a seal around the laryngeal inlet. The cuff can be inflatable, and in most variants it surrounds a hollow interior space or lumen of the mask, the at least one airway tube opening into the lumen. U.S. Pat. No. 4,509,514 is one of the many publications that describe laryngeal mask airway devices such as this. It is relatively easy to insert a laryngeal mask airway device into a patient and thereby establish an airway. Also, the laryngeal mask airway device is a “forgiving” device in that even if it is inserted improperly, it still tends to establish an airway. Accordingly, the laryngeal mask airway device is often thought of as a “life saving” device. Also, the laryngeal mask airway device may be inserted with only relatively minor manipulation of the patient's head, neck and jaw. Further, the laryngeal mask airway device provides ventilation of the patient's lungs without requiring contact with the sensitive inner lining of the trachea and the size of the airway established is typically significantly larger than the size of the airway established with an endotracheal tube. Also, the laryngeal mask airway device does not interfere with coughing to the same extent as endotracheal tubes. Largely due to these advantages, the laryngeal mask airway device has enjoyed increasing popularity in recent years.

For convenience of expression, the term “fully inserted configuration” shall be used to refer to a laryngeal mask airway device that has been inserted into a patient and has the following characteristics: (1) the patient end of the mask portion is pressed against the patient's normally closed oesophageal sphincter; (2) the cuff is inflated and forms a seal around the patient's glottis opening; and (3) the airway tube extends from a proximal end located outside the patient's mouth to a distal portion that is coupled to the mask portion, the tube extending through the patient's mouth and the patient's natural upper airway so that the device provides a sealed airway extending from the tube's proximal end to the patient's lungs.

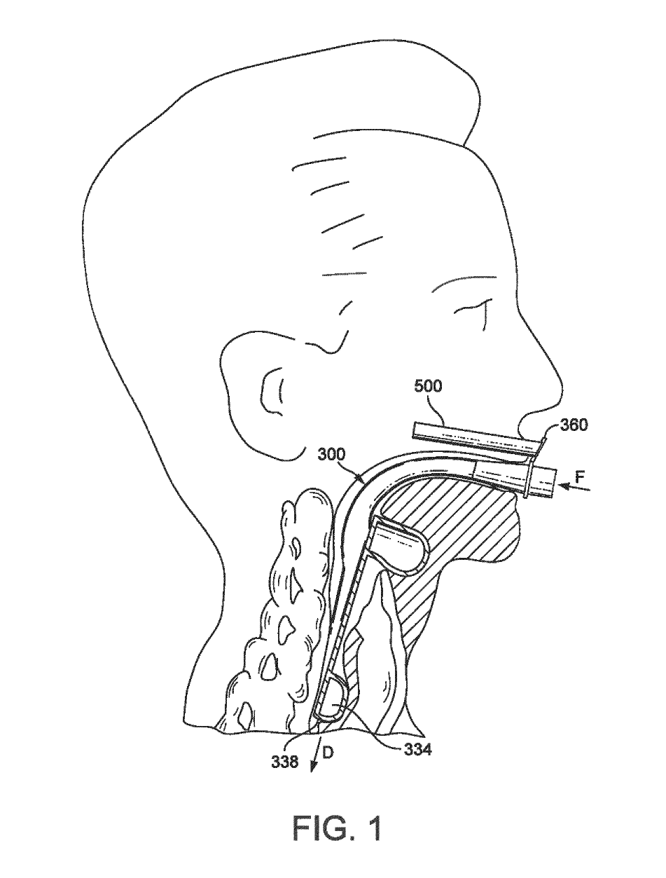

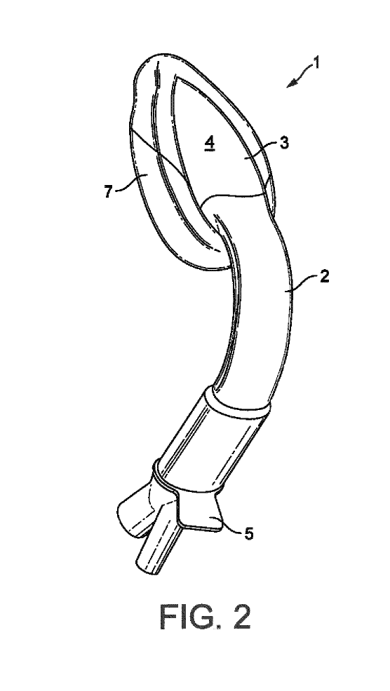

FIG. 1 shows a prior art disposable laryngeal mask airway device (300) as described in, for example, EP 2165727, comprising a tab (360) disposed on the airway tube, wherein the tab is located so that adhesive tape (500) may be attached to the tab and the patient's cheeks. The tape applies a force that biases the device generally into a patient and, in particular, biases the distal end of the device against the patient's oesophageal sphincter. Such an arrangement allows the device to remain more stably in the inserted configuration and reduces the likelihood that regurgitated material will be aspirated into the patient's lungs. In particular, the tab (360) facilitates maintaining firm contact between the distal end (338) of the cuff (334) and the patient's normally closed oesophageal sphincter.

FIG. 2 shows a prior art disposable laryngeal mask airway device (1) as described in, for example, WO 2006/125990, comprising a fixation tab (5) affixed to the proximal end of the airway tube.

Although prior art disposable laryngeal mask airway devices have performed well, there remains a need for providing improved devices. In particular, there remains a need for providing a disposable laryngeal mask airway device that more reliably remains in the fully inserted configuration once the device has been inserted in a patient. In addition, there is a need to provide a more secure means for attaching a laryngeal mask airway device to a patient. There is also a need for providing a disposable laryngeal mask airway device comprising a fixation device that can be attached to different positions along the longitudinal length of the airway tube, to provide more flexibility in using the device for patients having different sizes of airways.

According to a first aspect, there is provided a fixation device for use with a laryngeal mask airway device, the fixation device comprising releasable locking means for releasably securing the fixation device to the laryngeal mask airway device.

Preferably, the releasable locking means is configured such that in a first configuration, the fixation device may be secured to the laryngeal mask airway device and locked in place, and in a second configuration, the fixation device may be unlocked and removed from the laryngeal mask airway device.

An advantage of the present invention is that in the locked configuration, the fixation device assists in holding the laryngeal mask airway device in the fully inserted configuration once it has been inserted in a patient. Furthermore, once the laryngeal mask airway device has been removed from the patient's airway, the fixation device may be unlocked and removed from the laryngeal mask airway device. The use of a fixation device having releasable locking means allows the fixation device to be used with more than one laryngeal mask airway device, thus allowing for greater flexibility. The fixation device of the present invention may also advantageously be used with a laryngeal mask device in patients having different sizes of airways.

Preferably, the releasable locking means comprises first and second parts, the first and second parts being mutually engageable with one another. Preferably, the releasable locking means comprises male and female parts, wherein the male part is mutually engageable with the female part. The provision of such mutually engageable male and female parts assists in the locking and/or unlocking of the fixation device to facilitate attachment of the fixation device to an outer surface of a laryngeal mask airway device and/or removal of the fixation device from the outer surface of the laryngeal mask airway device.

Preferably, the first and second parts comprise a hook and aperture combination, wherein the hook is releasably engageable with the aperture. The provision of a hook and aperture combination provides a means of easily locking and/or unlocking the fixation device, thus assisting in securing the fixation device to the outer surface of a laryngeal mask airway device when the hook is engaged with the aperture and/or removing the fixation device from the outer surface of a laryngeal mask airway device when the hook and aperture combination are disengaged. In one embodiment, the releasable locking means may be biased towards the locked configuration. In one embodiment, the first and second parts comprise a hook and recess combination, wherein the hook is releasably engageable with the recess to facilitate locking and/or unlocking of the fixation device.

Preferably, the releasable locking means comprises a lever to facilitate the locking and/or unlocking of the first and second parts. Preferably, the lever is provided to facilitate locking and/or unlocking of the male and female parts. Preferably, the lever extends from the first part, preferably the male part, of the releasable locking means, such that the user may use the lever to lock and/or unlock the fixation device. Preferably, the lever extends outwards from the releasable locking means in an arcuate shape. Advantageously, the arcuate shape allows the user to place their finger on the lever to facilitate the locking and/or unlocking of the first and second parts. Typically, when using the fixation device of the present invention, the user may need to reposition the fixation device with respect to the proximal end of the airway device, particularly in cases where the user may mistakenly position the fixation device in the wrong position with respect to the airway device, for example, where the user has a lack of experience in determining the optimal position of the fixation device. Thus, the provision of a lever advantageously assists the user in positioning and/or repositioning the first and second parts during locking and/or unlocking of the releasable locking means of the fixation device.

Preferably, the fixation device is shaped to correspond with the shape of the proximal end of the laryngeal mask airway device. Preferably, the inner surface of the fixation device corresponds to the shape of the outer surface of the proximal end of the laryngeal mask airway device. Preferably, the fixation device is shaped to correspond with the shape of a biteblock of the laryngeal mask airway device. Preferably, the inner surface of the fixation device corresponds to the shape of the outer surface of the biteblock of the laryngeal mask airway device. In one embodiment, the fixation device may comprise a ring shape. Preferably, the ring shape of the fixation device comprises a break, wherein the releasable locking means is provided. Preferably, the lever is positioned adjacent to the break.

Preferably, the fixation device is secured to an outer surface of the laryngeal mask airway device, preferably at the proximal end thereof: Preferably, the fixation device is secured to the outer surface of a biteblock of the laryngeal mask airway device. Preferably, the proximal end of the laryngeal mask airway device comprises an internal feature that strengthens this region of the device. Thus, securing the fixation device to the proximal end of the laryngeal mask airway device has the advantage of avoiding crushing of the laryngeal mask airway device. In one embodiment, the internal strengthening feature may comprise a biteblock. In one embodiment, the internal strengthening feature may comprise the material joining an airway tube and a gastro tube provided within the laryngeal mask airway device, i.e. at the junction between the airway tube and the gastro tube.

In one embodiment, the releasable locking means may be provided along one side of the fixation device.

Preferably, the fixation device comprises a strap mount around which a strap can be releasably fixed for securing the laryngeal mask airway device to a patient. The provision of a fixation device comprising a strap mount assists in more reliably securing the laryngeal mask airway device in the fully inserted configuration once the device has been inserted in a patient. Preferably, the strap is between 500 and 700 mm in length, more preferably between 550 and 650 mm in length, and most preferably around 650 mm in length. Advantageously, the provision of a strap which is around 650 mm in length allows the strap to be used with patients of a variety of sizes, from small to larger patients. The use of a strap with a length of around 650 mm overcomes the problems that may be associated with use of a shorter strap length which may be too small for some patients.

It is preferred that the strap mount comprises at least one mount bar. Typically, the strap mount comprises two mount bars. Such arrangement assists in attaching one or more straps to the strap mount, to assist in securing the laryngeal mask airway device to a patient.

Preferably, the at least one mount bar is disposed between two arms which extend in a direction substantially perpendicular to the longitudinal axis of the device, wherein a mount bar is disposed on either side of the device. Thus, the laryngeal mask airway device is more reliably held in position when in the fully inserted configuration.

Preferably, the fixation device further comprises at least one tab. In a preferred embodiment, the fixation device comprises two tabs. Preferably, the fixation device comprises two tabs, each tab being provided on a mount bar. It is preferred that the at least one tab is shaped to correspond with an aperture provided on a strap, such that the at least one tab is provided to hold the strap in place when the device is inserted within a patient. Preferably, the at least one tab has dimensions that allow it to be robust, preventing breakage of the at least one tab and facilitating attachment of the strap to the at least one tab.

Preferably, the strap comprises a plurality of holes for attachment to the tab and/or to the strap mount. Preferably, the strap comprises between 10 and 30, preferably between 15 and 25, and most preferably around 20 holes along the length thereof. Preferably, each hole has a diameter which is sized to correspond with the size of the tab. Preferably, each hole has a diameter of between 2 and 6 mm, preferably between 3 and 5 mm, and most preferably around 4 mm.

Preferably, the fixation device comprises a plastics material. In one embodiment, the fixation device may comprise polyvinyl chloride or silicone.

It is preferred that the fixation device further comprises means for repositioning the fixation device with respect to the laryngeal mask airway device at one or more different positions. Thus, the fixation device can be secured in a plurality of positions with respect to the outer surface of the laryngeal mask airway device, thus allowing the device to be adapted for patients having different sizes of airways.

Preferably, the means for repositioning comprises a male part provided on the fixation device which is mutually engageable with a female part provided on an outer surface of the laryngeal mask airway device. In another embodiment, the means for repositioning comprises a female part provided on the fixation device which is mutually engageable with a male part provided on an outer surface of the laryngeal mask airway device. Thus, the fixation device may be releasably secured at different positions on the outer surface of the laryngeal mask airway device.

Preferably, the means for repositioning comprises at least one ridge provided on an inner surface of the fixation device. Typically, the means for repositioning comprises two ridges.

Preferably, the at least one ridge engages with at least one recess provided on an outer surface of the laryngeal mask airway device.

Typically, the at least one recess is provided on an outer surface of a biteblock of the laryngeal mask airway device.

It is preferred that the at least one ridge is mutually engageable with the at least one recess. Thus, the fixation device may be releasably secured at different positions of the outer surface of the laryngeal mask airway device.

In another embodiment, the means for repositioning may comprise at lease one ridge provided on an outer surface of the laryngeal mask airway device which may be mutually engageable with at least one recess provided on an inner surface of the fixation device.

Preferably, the at least one recess extends along a side of the biteblock in a direction substantially perpendicular with the longitudinal axis of the laryngeal mask airway device.

Preferably, the fixation device is movable between a first position and a second position along the longitudinal length of the laryngeal mask airway device and is releasably securable thereto.

According to a second aspect, there is provided a fixation device for use with a laryngeal mask airway device, the fixation device comprising releasable repositioning means for releasably securing the fixation device to the laryngeal mask airway device at a plurality of different positions. Such arrangement advantageously allows the device to be adapted for patients having different sizes of airways.

Preferably, the means for repositioning comprises a male part provided on the fixation device which is mutually engageable with a female part provided on an outer surface of the laryngeal mask airway device. In another embodiment, the means for repositioning comprises a female part provided on the fixation device which is mutually engageable with a male part provided on an outer surface of the laryngeal mask airway device. Thus, the fixation device may be releasably secured at different positions on the outer surface of the laryngeal mask airway device.

Preferably, the means for repositioning comprises at least one ridge provided on an inner surface of the fixation device. Typically, the means for repositioning comprises two ridges.

Preferably, the at least one ridge engages with at least one recess provided on an outer surface of the laryngeal mask airway device.

Typically, the at least one recess is provided on an outer surface of a biteblock of the laryngeal mask airway device.

Preferably, the at least one ridge is mutually engageable with the at least one recess. Thus, the fixation device may be releasably secured at different positions with respect to the outer surface of the laryngeal mask airway device.

In another embodiment, the means for repositioning may comprise at lease one ridge provided on an outer surface of the laryngeal mask airway device which may be mutually engageable with at least one recess provided on an inner surface of the fixation device.

It is preferred that the at least one recess extends along a side of the biteblock in a direction substantially perpendicular to the longitudinal axis of the laryngeal mask airway device.

Preferably, the fixation device is movable to between a first and second position along the longitudinal length of the laryngeal mask airway device and releasably securable thereto.

It is preferred that the fixation device further comprises releasable locking means comprising first and second parts, the first and second parts being mutually engageable with one another.

Preferably, the releasable locking means is configured such that in a first configuration, the fixation device may be secured to the laryngeal mask airway device and locked in place, and in a second configuration, the fixation device may be unlocked and removed from the laryngeal mask airway device. Preferably, the fixation device is secured to an outer surface of the laryngeal mask airway device, preferably at the proximal end thereof. Preferably, the proximal end of the laryngeal mask airway device comprises an internal feature that strengthens this region of the device. Thus, securing the fixation device to the proximal end of the laryngeal mask airway device has the advantage of avoiding crushing of the laryngeal mask airway device.

The use of a fixation device comprising releasable locking means has the advantage that, in the locked configuration, it assists in holding the laryngeal mask airway device in the fully inserted configuration once the laryngeal mask airway device has been inserted in a patient. Once the laryngeal mask airway device has been removed from the patient's airway, the fixation device may be unlocked and removed from the laryngeal mask airway device. The use of a fixation device having releasable locking means allows the fixation device to be used with more than one laryngeal mask airway device, thus allowing for greater flexibility.

Preferably, the releasable locking means comprises first and second parts, the first and second parts being mutually engageable with one another. Preferably, the releasable locking means comprises male and female parts, wherein the male part is mutually engageable with the female part. The provision of such mutually engageable male and female parts assists in the locking and/or unlocking of the fixation device to facilitate attachment of the fixation device to an outer surface of a laryngeal mask airway device and/or removal of the fixation device from the outer surface of the laryngeal mask airway device.

Preferably, the first and second parts comprise a hook and aperture combination, wherein the hook is releasably engageable with the aperture. Such arrangement facilitates the locking and/or unlocking of the fixation device, thus assisting in securing the fixation device to a laryngeal mask airway device when the hook is engaged with the aperture and/or removing the fixation device from the laryngeal mask airway device when the hook and aperture combination are disengaged. In one embodiment, the first and second parts comprise a hook and recess combination, wherein the hook is releasably engageable with the recess.

Preferably, the releasable locking means comprises a lever to facilitate the locking and/or unlocking of the first and second parts. Preferably, the lever is provided to facilitate locking and/or unlocking of the male and female parts. Preferably, the lever extends from the first part, preferably the male part, of the releasable locking means, such that the user may use the lever to lock and/or unlock the fixation device. Preferably, the lever extends outwards from the releasable locking means in an arcuate shape. Advantageously, the arcuate shape allows the user to place their finger on the lever to facilitate the locking and/or unlocking of the first and second parts. Typically, when using the fixation device of the present invention, the user may need to reposition the fixation device with respect to the proximal end of the airway device, particularly in cases where the user may mistakenly position the fixation device in the wrong position with respect to the airway device, for example, where the user has a lack of experience in determining the optimal position of the fixation device. Thus, the provision of a lever advantageously assists the user in positioning and/or repositioning the first and second parts during locking and/or unlocking of the releasable locking means of the fixation device.

Preferably, the fixation device is shaped to correspond with the shape of the proximal end of the laryngeal mask airway device. Preferably, the inner surface of the fixation device corresponds to the shape of the outer surface of the proximal end of the laryngeal mask airway device. Preferably, the fixation device is shaped to correspond with the shape of a biteblock of the laryngeal mask airway device. Preferably, the inner surface of the fixation device corresponds to the shape of the outer surface of the biteblock of the laryngeal mask airway device. In one embodiment, the fixation device may comprise a ring shape. Preferably, the ring shape of the fixation device comprises a break, wherein the releasable locking means is provided. Preferably, the lever is positioned adjacent the break.

In one embodiment, the releasable locking means may be provided along one side of the fixation device.

Typically, the fixation device comprises a strap mount around which a strap can be removably fixed for securing the laryngeal mask airway device to a patient. The provision of a fixation device comprising a strap mount assists in more reliably securing the laryngeal mask airway device in the fully inserted configuration once the device has been inserted in a patient. Preferably, the strap has a length of between 500 and 700 mm, preferably between 550 mm and 650 mm, and most preferably around 650 mm in length. Advantageously, the provision of a strap which is around 650 mm in length allows the strap to be used with patients of a variety of sizes, from small to larger patients. The use of a strap with a length of around 650 mm overcomes the problems that may be associated with use of a shorter strap length which may be too small for some patients.

Preferably, the fixation device comprises at least one mount bar. It is preferred that the fixation device comprises two mount bars. The provision of a strap mount having one or two mount bars assists in attaching one or more straps to the strap mount, to assist in securing the laryngeal mask airway device to a patient.

Preferably, the at least one bar is disposed between two arms which extend in a direction substantially perpendicular to the longitudinal axis of the device, wherein a mount bar is disposed on either side of the laryngeal mask airway device. Typically, the at least one mount bar is disposed at the proximal end of the laryngeal mask airway device (i.e. the end of the device that is nearest the patient). Thus, the laryngeal mask airway device is more reliably held in position when inserted within a patient.

Preferably, the fixation device further comprises at least one tab. In a preferred embodiment, the fixation device comprises two tabs. Preferably, the fixation device comprises two tabs, each tab being provided on a mount bar. It is preferred that the at least one tab is shaped to correspond with an aperture provided on a strap, such that the at least one tab is provided to hold the strap in place when the device is inserted within a patient. Preferably, the at least one tab has dimensions that allow it to be robust, preventing breakage of the at least one tab and facilitating attachment of the strap to the at least one tab.

Preferably, the strap comprises a plurality of holes for attachment to the tab and/or to the strap mount. Preferably, the strap comprises between 10 and 30, preferably between 15 and 25, and most preferably around 20 holes along the length thereof. Preferably, each hole has a diameter which is sized to correspond with the size of the tab. Preferably, each hole has a diameter of between 2 and 6 mm, preferably between 3 and 5 mm, and most preferably around 4 mm.

Preferably, the fixation device comprises a plastics material. In one embodiment, the fixation device comprises polyvinyl chloride or silicone.

According to a third aspect, there is provided a laryngeal mask airway device, comprising at least one airway tube and a mask carried at one end of the at least one airway tube, the mask having a distal end and a proximal end and a peripheral formation capable of conforming to, and of fitting within, the actual and potential space behind the larynx of the patient so as to form a seal around the circumference of the laryngeal inlet, the peripheral formation surrounding a hollow interior space or lumen of the mask and the at least one airway tube opening into the lumen of the mask, the peripheral formation establishing separation between a laryngeal chamber side and a pharyngeal side, the device further comprising a fixation device comprising releasable locking means for releasably securing the strap mount to the device.

Preferably, the releasable locking means is configured such that in a first configuration, the fixation device may be secured to the laryngeal mask airway device and locked in place, and in a second configuration, the fixation device may be unlocked and removed from the laryngeal mask airway device. Preferably, the fixation device is secured to an outer surface of the laryngeal mask airway device, preferably at the proximal end thereof. Preferably, the proximal end of the laryngeal mask airway device comprises an internal feature that strengthens this region of the device. Thus, securing the fixation device to the proximal end of the laryngeal mask airway device has the advantage of avoiding crushing of the laryngeal mask airway device.

Thus, in a first configuration, the fixation device may be secured to the laryngeal mask airway device and locked in place, and in a second configuration, the fixation device may be unlocked and removed from the laryngeal mask airway device. An advantage of the present invention is that in the locked configuration, the fixation device assists in holding the laryngeal mask airway device in the fully inserted configuration once it has been inserted in a patient. Furthermore, once the laryngeal mask airway device has been removed from the patient's airway, the fixation device may be unlocked and removed from the laryngeal mask airway device. The use of a fixation device having releasable locking means allows the fixation device to be used with more than one laryngeal mask airway device, thus allowing for greater flexibility, and advantageously provides for secure attachment of the laryngeal mask airway device to a patient.

Preferably, the releasable locking means comprises first and second parts, the first and second parts being mutually engageable with one another. Preferably, the releasable locking means comprises male and female parts, wherein the male part is mutually engageable with the female part. The provision of such mutually engageable male and female parts assists in the locking and/or unlocking of the fixation device to facilitate attachment of the fixation device to an outer surface of a laryngeal mask airway device and/or removal of the fixation device from the outer surface of the laryngeal mask airway device.

Preferably, the first and second parts comprise a hook and aperture combination, wherein the hook is releasably engageable with the aperture. The provision of a hook and aperture combination provides a means of easily locking and/or unlocking the fixation device, thus assisting in securing the fixation device to the outer surface of a laryngeal mask airway device when the hook is engaged with the aperture and/or removing the fixation device from the outer surface of a laryngeal mask airway device when the hook and aperture combination are disengaged. In one embodiment, the releasable locking means may be biased towards the locked configuration. In one embodiment, the first and second parts comprise a hook and recess combination, wherein the hook is releasably engageable with the recess.

Preferably, the releasable locking means comprises a lever to facilitate the locking and/or unlocking of the first and second parts. Preferably, the lever is provided to facilitate locking and/or unlocking of the male and female parts. Preferably, the lever extends from the first part, preferably the male part, of the releasable locking means, such that the user may use the lever to lock and/or unlock the fixation device. Preferably, the lever extends outwards from the releasable locking means in an arcuate shape. Advantageously, the arcuate shape allows the user to place their finger on the lever to facilitate the locking and/or unlocking of the first and second parts. Typically, when using the fixation device of the present invention, the user may need to reposition the fixation device with respect to the proximal end of the airway device, particularly in cases where the user may mistakenly position the fixation device in the wrong position with respect to the airway device, for example, where the user has a lack of experience in determining the optimal position of the fixation device. Thus, the provision of a lever advantageously assists the user in positioning and/or repositioning the first and second parts during locking and/or unlocking of the releasable locking means of the fixation device.

Preferably, the fixation device is shaped to correspond with the shape of the proximal end of the laryngeal mask airway device. Preferably, the inner surface of the fixation device corresponds to the shape of the outer surface of the proximal end of the laryngeal mask airway device. Preferably, the fixation device is shaped to correspond with the shape of a biteblock of the laryngeal mask airway device. Preferably, the inner surface of the fixation device corresponds to the shape of the outer surface of the biteblock of the laryngeal mask airway device. In one embodiment, the fixation device may comprise a ring shape. Preferably, the ring shape of the fixation device comprises a break, wherein the releasable locking means is provided. Preferably, the lever is positioned adjacent the break.

In one embodiment, the releasable locking means may be provided along one side of the fixation device.

Preferably, the fixation device comprises a strap mount around which a strap can be removably fixed for securing the laryngeal mask airway device to a patient. The provision of a fixation device comprising a strap mount assists in more reliably securing the laryngeal mask airway device in the fully inserted configuration once the device has been inserted in a patient. Preferably, the strap has a length of between 500 and 700 mm, preferably between 550 mm and 650 mm, and most preferably around 650 mm in length. Advantageously, the provision of a strap which is around 650 mm in length allows the strap to be used with patients of a variety of sizes, from small to larger patients. The use of a strap with a length of around 650 mm advantageously overcomes the problems that may be associated with use of a shorter strap length which may be too small for some patients.

It is preferred that the strap mount comprises at least one mount bar. Typically, the strap mount comprises two mount bars. Such arrangement assists in attaching one or more straps to the strap mount, to assist in securing the laryngeal mask airway device to a patient.

Preferably, the at least one mount bar is disposed between two arms which extend in a direction substantially perpendicular to the longitudinal axis of the device, wherein a mount bar is disposed on either side of the device. Thus, the laryngeal mask airway device is more reliably held in position when in the fully inserted configuration.

Preferably, the fixation device further comprises at least one tab. In a preferred embodiment, the fixation device comprises two tabs. Preferably, the fixation device comprises two tabs, each tab being provided on a mount bar. It is preferred that the at least one tab is shaped to correspond with an aperture provided on a strap, such that the at least one tab is provided to hold the strap in place when the device is inserted within a patient. Preferably, the at least one tab has dimensions that allow it to be robust, preventing breakage of the at least one tab and facilitating attachment of the strap to the at least one tab.

Preferably, the strap comprises a plurality of holes for attachment to the tab and/or to the strap mount. Preferably, the strap comprises between 10 and 30, preferably between 15 and 25, and most preferably around 20 holes along the length thereof. Preferably, each hole has a diameter which is sized to correspond with the size of the tab. Preferably, each hole has a diameter of between 2 and 6 mm, preferably between 3 and 5 mm, and most preferably around 4 mm.

Preferably, the fixation device comprises a plastics material. In one embodiment, the fixation device may comprise polyvinyl chloride or silicone.

Preferably, the fixation device comprises releasable repositioning means for releasably securing the fixation device to the laryngeal mask airway device at a plurality of different positions. Such an arrangement assists in use of the laryngeal mask airway device in patients having different sizes of airways.

Preferably, the means for repositioning comprises a male part provided on the fixation device which is mutually engageable with a female part provided on an outer surface of the laryngeal mask airway device. In another embodiment, the means for repositioning comprises a female part provided on the fixation device which is mutually engageable with a male part provided on an outer surface of the laryngeal mask airway device. Thus, the fixation device may be releasably secured at different positions on the outer surface of the laryngeal mask airway device.

Preferably, the means for repositioning comprises at least one ridge provided on an inner surface of the fixation device. Typically, the means for repositioning comprises two ridges.

Preferably, the at least one ridge engages with at least one recess provided on an outer surface of the laryngeal mask airway device.

Typically, the at least one recess is provided on an outer surface of a biteblock of the laryngeal mask airway device.

Preferably, the at least one ridge is mutually engageable with the at least one recess. Thus, the fixation device may be releasably secured at different positions with respect to the outer surface of the laryngeal mask airway device.

In another embodiment, the means for repositioning may comprise at lease one ridge provided on an outer surface of the laryngeal mask airway device which may be mutually engageable with at least one recess provided on an inner surface of the fixation device.

It is preferred that the at least one recess extends along a side of the biteblock in a direction substantially perpendicular to the longitudinal axis of the laryngeal mask airway device.

Preferably, the fixation device is movable between a first and second position along the longitudinal length of the laryngeal mask airway device and releasably securable thereto.

Preferably, the laryngeal mask airway device further comprises a conduit adapted for passage of an endoscope into the oesophagus of a patient when the mask is in place, the conduit including a distal end for cooperation with the oesophageal sphincter of the patient, the conduit having a laryngeal side, a pharyngeal side, and with respect thereto, a right side and a left side, and an outlet. Thus, the laryngeal mask airway device of the present invention has the combined benefits of establishing an airway within a patient and protecting the airway from regurgitation or vomiting that might be caused by endoscopy by providing a close cooperation between the oesophageal sphincter of the patient and the distal end of the conduit. Thus, the device of the present invention advantageously assists in the safe and accurate insertion of an endoscope within the oesophagus of a patient, whilst at the same time establishing an airway within the patient.

Preferably, at the outlet, the width between the left side and the right side is smaller than the width of the conduit.

Preferably, at the outlet, the pharyngeal side is terminated proximally of the laryngeal side.

Preferably, at the outlet, the width of the conduit wall is reduced. Typically, the width reduction is at the left and the right side of the outlet.

It is preferred that the distal end of the conduit is provided at an angle of about 30 degrees, relative to the longitudinal axis of the conduit.

Preferably, the conduit includes an internal sleeve at its proximal end.

Preferably, the laryngeal mask airway device further comprises a connector. Typically, the sleeve is integrally formed with the connector.

Preferably, the conduit has a large bore diameter. Typically, the conduit has a diameter of between about 5 and 25 mm, more typically between about 10 and 20 mm, and most typically about 15 mm, depending on the size of the laryngeal mask airway device. The diameter of the conduit advantageously allows the passage of an endoscope through the conduit. Thus, the conduit typically forms an “operating channel” through which a surgeon can view the upper gastrointestinal tract of a patient. In addition, the diameter of the conduit may be varied depending on the anatomy of a patient. Preferably, the radial wall thickness of the conduit is between about 1 to 2 mm.

Typically, the laryngeal mask airway device of the present invention is inserted into the upper oesophageal sphincter by means of the tip of the cuff, which provides guided insertion of the device. Furthermore, the distal flexibility of the tip of the device may assist in tracking the posterior curvature of the throat of a patient upon insertion of the device and reduces trauma to the throat of the patient.

The use of a laryngeal mask airway device in accordance with the present invention in combination with an endoscope is safer than use of the endoscope alone and advantageously allows the endoscope to be inserted within the oesophagus of a patient for a longer period of time.

Typically, the peripheral formation may be inflatable. Preferably, the peripheral formation comprises an inflatable cuff. Typically, the cuff is capable of conforming to and fitting within the actual and potential space behind the larynx of a patient so as to form a seal around the laryngeal inlet. Typically, the cuff extends from a proximal end to a distal end.

According to a fourth aspect, there is provided a laryngeal mask airway device, comprising at least one airway tube and a mask carried at one end of the at least one airway tube, the mask having a distal end and a proximal end and a peripheral formation capable of conforming to, and of fitting within, the actual and potential space behind the larynx of the patient so as to form a seal around the circumference of the laryngeal inlet, the peripheral formation surrounding a hollow interior space or lumen of the mask and the at least one airway tube opening into the lumen of the mask, the peripheral formation establishing separation between a laryngeal chamber side and a pharyngeal side, the device further comprising a fixation device comprising releasable repositioning means for releasably securing the fixation device to the laryngeal mask airway device at a plurality of different positions. Such arrangement advantageously allows the device to be adapted for patients having different sizes of airways.

Preferably, the means for repositioning comprises a male part provided on the fixation device which is mutually engageable with a female part provided on an outer surface of the laryngeal mask airway device. In another embodiment, the means for repositioning comprises a female part provided on the fixation device which is mutually engageable with a male part provided on an outer surface of the laryngeal mask airway device. Thus, the fixation device may be releasably secured at different positions on the outer surface of the laryngeal mask airway device.

Preferably, the means for repositioning comprises at least one ridge provided on an inner surface of the fixation device. Typically, the means for repositioning comprises two ridges.

Preferably, the at least one ridge engages with at least one recess provided on an outer surface of the laryngeal mask airway device.

Typically, the at least one recess is provided on an outer surface of a biteblock of the laryngeal mask airway device.

Preferably, the at least one ridge is mutually engageable with the at least one recess. Thus, the fixation device may be releasably secured at different positions with respect to the outer surface of the laryngeal mask airway device.

In another embodiment, the means for repositioning may comprise at lease one ridge provided on an outer surface of the laryngeal mask airway device which may be mutually engageable with at least one recess provided on an inner surface of the fixation device.

It is preferred that the at least one recess extends along a side of the biteblock in a direction substantially perpendicular to the longitudinal axis of the laryngeal mask airway device.

Preferably, the fixation device is movable to between a first and second position along the longitudinal length of the laryngeal mask airway device and releasably securable thereto.

It is preferred that the fixation device further comprises releasable locking means comprising first and second parts, the first and second parts being mutually engageable with one another.

Preferably, the releasable locking means is configured such that in a first configuration, the fixation device may be secured to the laryngeal mask airway device and locked in place, and in a second configuration, the fixation device may be unlocked and removed from the laryngeal mask airway device. Preferably, the fixation device is secured to an outer surface of the laryngeal mask airway device, preferably at the proximal end thereof. Preferably, the proximal end of the laryngeal mask airway device comprises an internal feature that strengthens this region of the device. Thus, securing the fixation device to the proximal end of the laryngeal mask airway device has the advantage of avoiding crushing of the laryngeal mask airway device.

The use of a fixation device comprising releasable locking means has the advantage that, in the locked configuration, it assists in holding the laryngeal mask airway device in the fully inserted configuration once the laryngeal mask airway device has been inserted in a patient. Once the laryngeal mask airway device has been removed from the patient's airway, the fixation device may be unlocked and removed from the laryngeal mask airway device. The use of a fixation device having releasable locking means allows the fixation device to be used with more than one laryngeal mask airway device, thus allowing for greater flexibility, and advantageously provides for secure attachment of the laryngeal mask airway device to a patient.

Preferably, the releasable locking means comprises first and second parts, the first and second parts being mutually engageable with one another. Preferably, the releasable locking means comprises male and female parts, wherein the male part is mutually engageable with the female part. The provision of such mutually engageable male and female parts assists in the locking and/or unlocking of the fixation device to facilitate attachment of the fixation device to an outer surface of a laryngeal mask airway device and/or removal of the fixation device from the outer surface of the laryngeal mask airway device.

Preferably, the first and second parts comprise a hook and aperture combination, wherein the hook is releasably engageable with the aperture. Such arrangement facilitates the locking and/or unlocking the fixation device, thus assisting in securing the fixation device to a laryngeal mask airway device when the hook is engaged with the aperture and/or removing the fixation device from the laryngeal mask airway device when the hook and aperture combination are disengaged. In one embodiment, the first and second parts comprise a hook and recess combination, wherein the hook is releasably engageable with the recess.

Preferably, the releasable locking means comprises a lever to facilitate the locking and/or unlocking of the first and second parts. Preferably, the lever is provided to facilitate locking and/or unlocking of the male and female parts. Preferably, the lever extends from the first part, preferably the male part, of the releasable locking means, such that the user may use the lever to lock and/or unlock the fixation device. Preferably, the lever extends outwards from the releasable locking means in an arcuate shape. Advantageously, the arcuate shape allows the user to place their finger on the lever to facilitate the locking and/or unlocking of the first and second parts. Typically, when using the fixation device of the present invention, the user may need to reposition the fixation device with respect to the proximal end of the airway device, particularly in cases where the user may mistakenly position the fixation device in the wrong position with respect to the airway device, for example, where the user has a lack of experience in determining the optimal position of the fixation device. Thus, the provision of a lever advantageously assists the user in positioning and/or repositioning the first and second parts during locking and/or unlocking of the releasable locking means of the fixation device.

Preferably, the fixation device is shaped to correspond with the shape of the proximal end of the laryngeal mask airway device. Preferably, the inner surface of the fixation device corresponds to the shape of the outer surface of the proximal end of the laryngeal mask airway device. Preferably, the fixation device is shaped to correspond with the shape of a biteblock of the laryngeal mask airway device. Preferably, the inner surface of the fixation device corresponds to the shape of the outer surface of the biteblock of the laryngeal mask airway device. In one embodiment, the fixation device may comprise a ring shape. Preferably, the ring shape of the fixation device comprises a break, wherein the releasable locking means is provided. Preferably, the lever is positioned adjacent the break.

In one embodiment, the releasable locking means may be provided along one side of the fixation device.

Typically, the fixation device comprises a strap mount around which a strap can be removably fixed for securing the laryngeal mask airway device to a patient. The provision of a fixation device comprising a strap mount assists in more reliably securing the laryngeal mask airway device in the fully inserted configuration once the device has been inserted in a patient. Preferably, the strap has a length of between 500 and 700 mm, preferably between 550 mm and 650 mm, and most preferably around 650 mm in length. Advantageously, the provision of a strap which is around 650 mm in length allows the strap to be used with patients of a variety of sizes, from small to larger patients. The use of a strap with a length of around 650 mm overcomes the problems that may be associated with use of a shorter strap length which may be too small for some patients.

Preferably, the fixation device comprises at least one mount bar. It is preferred that the fixation device comprises two mount bars. The provision of a strap mount having one or two mount bars assists in attaching one or more straps to the strap mount, to assist in securing the laryngeal mask airway device to a patient.

Preferably, the at least one bar is disposed between two arms which extend in a direction substantially perpendicular to the longitudinal axis of the device, wherein a mount bar is disposed on either side of the laryngeal mask airway device. Typically, the at least one mount bar is disposed at the proximal end of the laryngeal mask airway device (i.e. the end of the device that is nearest the patient). Thus, the laryngeal mask airway device is more reliably held in position when inserted within a patient.

Preferably, the fixation device further comprises at least one tab. In a preferred embodiment, the fixation device comprises two tabs. Preferably, the fixation device comprises two tabs, each tab being provided on a mount bar. It is preferred that the at least one tab is shaped to correspond with an aperture provided on a strap, such that the at least one tab is provided to hold the strap in place when the device is inserted within a patient. Preferably, the at least one tab has dimensions that allow it to be robust, preventing breakage of the at least one tab and facilitating attachment of the strap to the at least one tab.

Preferably, the strap comprises a plurality of holes for attachment to the tab and/or to the strap mount. Preferably, the strap comprises between 10 and 30, preferably between 15 and 25, and most preferably around 20 holes along the length thereof.

Preferably, each hole has a diameter which is sized to correspond with the size of the tab. Preferably, each hole has a diameter of between 2 and 6 mm, preferably between 3 and 5 mm, and most preferably around 4 mm.

Preferably, the fixation device comprises a plastics material. In one embodiment, the fixation device comprises polyvinyl chloride or silicone.

Preferably, the laryngeal mask airway device further comprises a conduit adapted for passage of an endoscope into the oesophagus of a patient when the mask is in place, the conduit including a distal end for cooperation with the oesophageal sphincter of the patient, the conduit having a laryngeal side, a pharyngeal side, and with respect thereto, a right side and a left side, and an outlet. Thus, the laryngeal mask airway device of the present invention has the combined benefits of establishing an airway within a patient and protecting the airway from regurgitation or vomiting that might be caused by endoscopy by providing a close cooperation between the oesophageal sphincter of the patient and the distal end of the conduit. Thus, the device of the present invention advantageously assists in the safe and accurate insertion of an endoscope within the oesophagus of a patient, whilst at the same time establishing an airway within the patient.

Preferably, at the outlet, the width between the left side and the right side is smaller than the width of the conduit.

Preferably, at the outlet, the pharyngeal side is terminated proximally of the laryngeal side.

Preferably, at the outlet, the width of the conduit wall is reduced. Typically, the width reduction is at the left and the right side of the outlet.

It is preferred that the distal end of the conduit is provided at an angle of about 30 degrees, relative to the longitudinal axis of the conduit.

Preferably, the conduit includes an internal sleeve at its proximal end.

Preferably, the laryngeal mask airway device further comprises a connector. Typically, the sleeve is integrally formed with the connector.

Preferably, the conduit has a large bore diameter. Typically, the conduit has a diameter of between about 5 and 25 mm, more typically between about 10 and 20 mm, and most typically about 15 mm, depending on the size of the laryngeal mask airway device. The diameter of the conduit advantageously allows the passage of an endoscope through the conduit. Thus, the conduit typically forms an “operating channel” through which a surgeon can view the upper gastrointestinal tract of a patient. In addition, the diameter of the conduit may be varied depending on the anatomy of a patient. Preferably, the radial wall thickness of the conduit is between about 1 to 2 mm.

Typically, the laryngeal mask airway device of the present invention is inserted into the upper oesophageal sphincter by means of the tip of the cuff, which provides guided insertion of the device. Furthermore, the distal flexibility of the tip of the device may assist in tracking the posterior curvature of the throat of a patient upon insertion of the device and reduces trauma to the throat of the patient.

The use of a laryngeal mask airway device in accordance with the present invention in combination with an endoscope is safer than use of the endoscope alone and advantageously allows the endoscope to be inserted within the oesophagus of a patient for a longer period of time.

Typically, the peripheral formation may be inflatable. Preferably, the peripheral formation comprises an inflatable cuff. Typically, the cuff is capable of conforming to and fitting within the actual and potential space behind the larynx of a patient so as to form a seal around the laryngeal inlet. Typically, the cuff extends from a proximal end to a distal end.

According to a fifth aspect, there is provided a kit comprising a laryngeal mask airway device and a fixation device according to the first and/or second aspects of the invention.

According to a sixth aspect, there is provided a method of releasably securing the fixation device of the first aspect to a laryngeal mask airway device.

According to a seventh aspect, there is provided a method of repositioning the fixation device of the second aspect to a laryngeal mask airway device at a plurality of different positions.

According to an eighth aspect, there is provided a method of making a laryngeal mask airway device according to the third and/or fourth aspect, comprising the steps of forming a laryngeal mask airway device, forming a fixation device according to the first and/or second aspect separately from the laryngeal mask airway device, wherein the fixation device is adapted to be releasably securable to the laryngeal mask airway device.

According to a ninth aspect, there is provided a method of inserting a laryngeal mask airway device into a patient, the method comprising the steps of inserting the mask to the required position and securing the mask in the required position by adjusting the position of the releasable fixation device of the first and/or second aspect relating to an airway tube of the device.

The invention will further be described by way of example and with reference to the following drawings, in which,

FIG. 1 shows a schematic side view of a prior art disposable laryngeal mask airway device comprising a tab to secure the device to a patient;

FIG. 2 shows a perspective view of a prior art disposable laryngeal mask airway device comprising a fixation device;

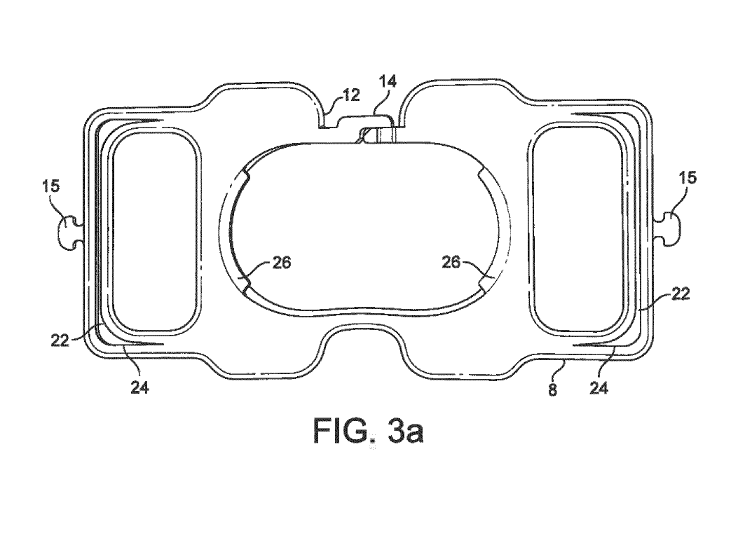

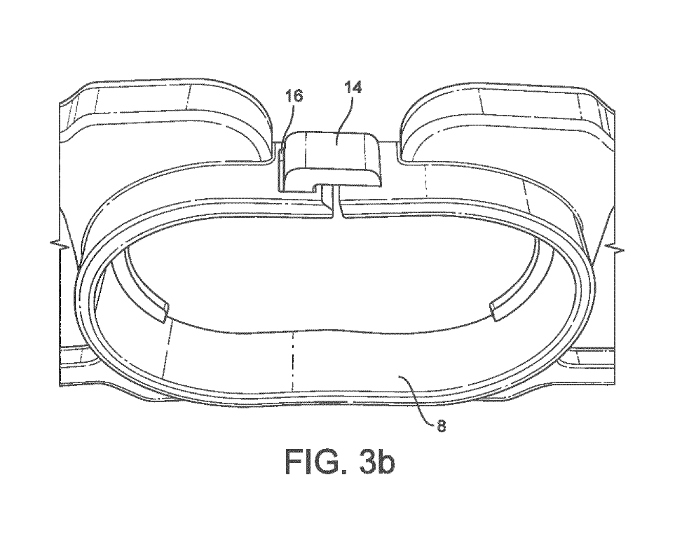

FIG. 3a shows a plan view of a fixation device according to an embodiment of the invention, wherein the fixation device is in a locked configuration;

FIG. 3b shows a perspective view of part of a fixation device according to an embodiment of the invention, wherein the fixation device is in a locked configuration;

FIG. 3c show a perspective side view of a fixation device according to an embodiment of the invention, wherein the fixation device is in a locked configuration;

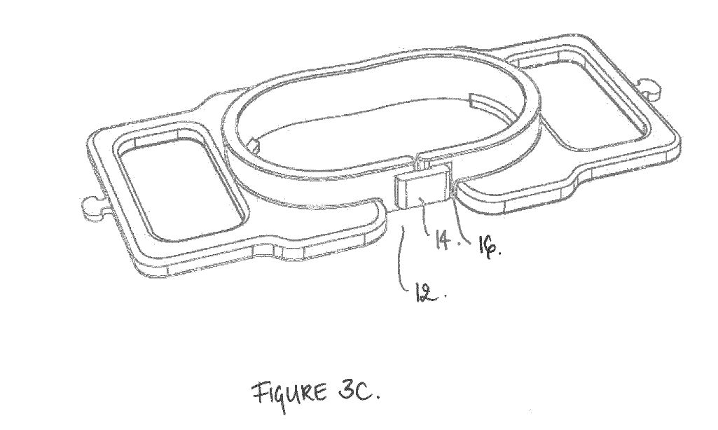

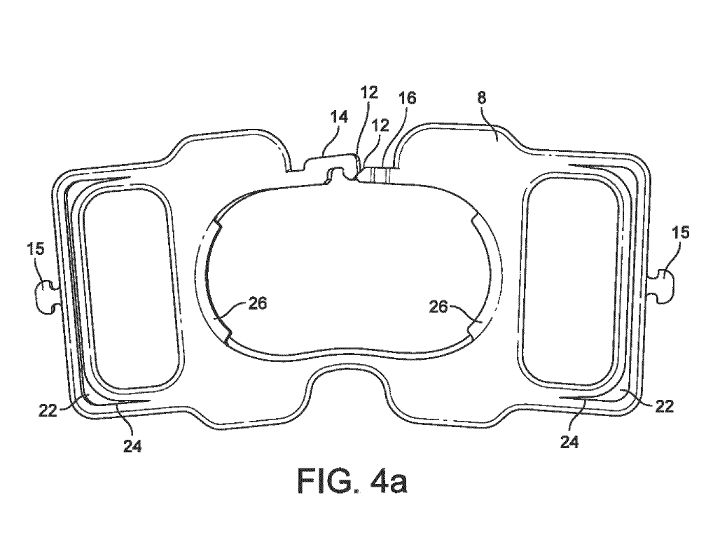

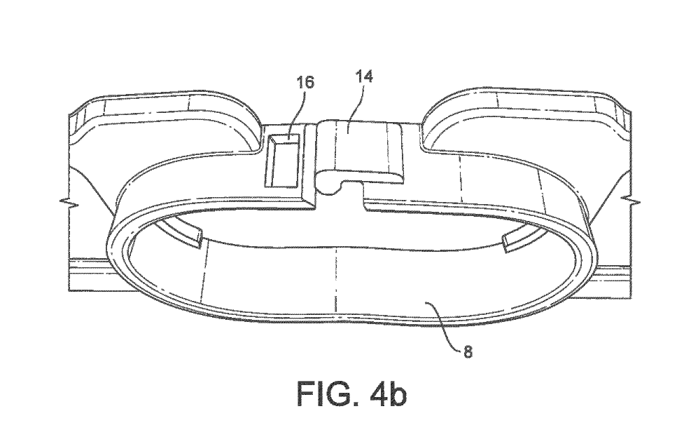

FIG. 4a shows a plan view of a fixation device according to an embodiment to the invention, wherein the fixation device is in the unlocked configuration;

FIG. 4b show a perspective side view of part of a fixation device according to an embodiment of the invention, wherein the fixation device is in an unlocked configuration;

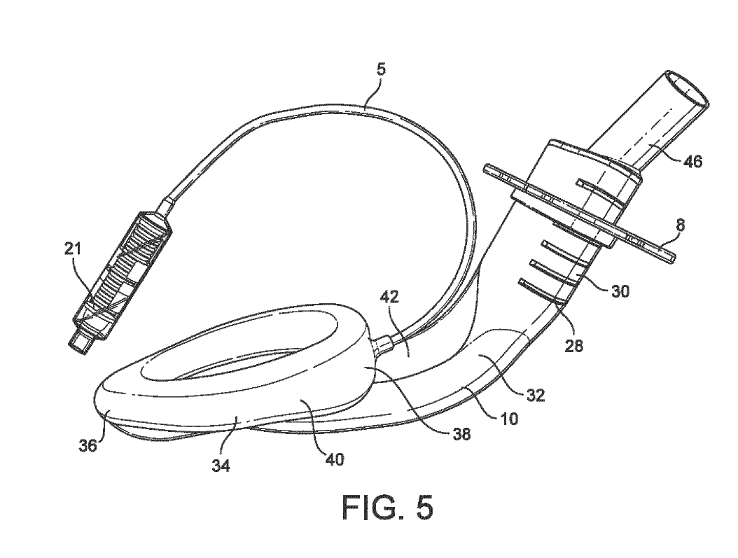

FIG. 5 is a perspective side view of a laryngeal mask airway device comprising a fixation device according to an embodiment of the invention;

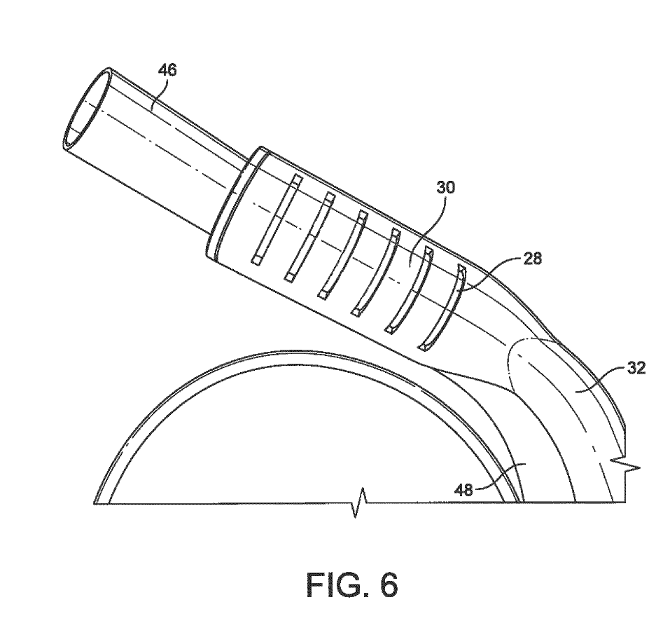

FIG. 6 is a perspective side view of the proximal end of a laryngeal mask airway device according to the invention without a fixation device;

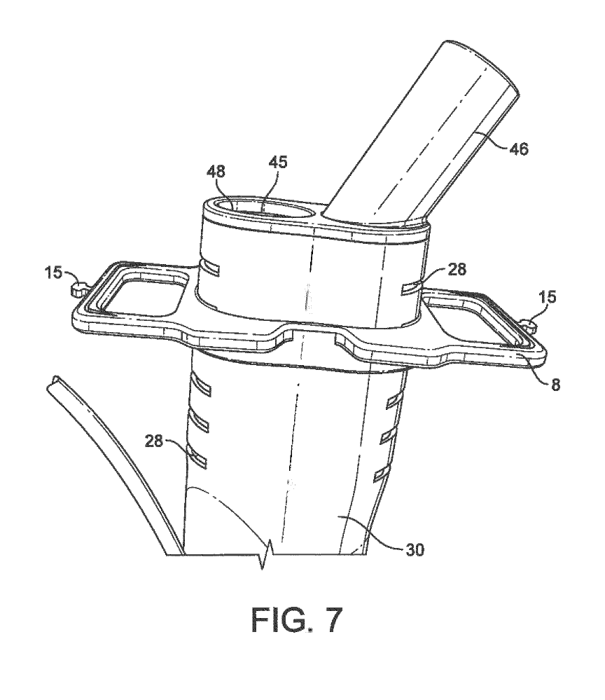

FIG. 7 is a perspective front view of the proximal end of a laryngeal mask airway device according to the invention with a fixation device;

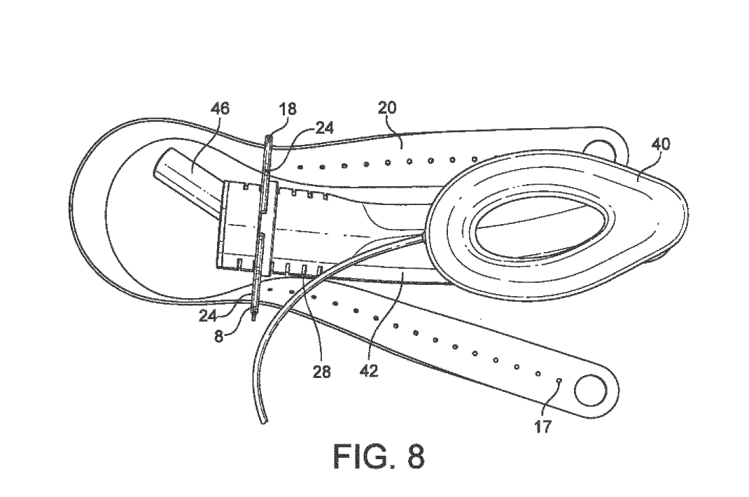

FIG. 8 is a perspective front view of a laryngeal mask airway device according to the invention in combination with a strap for securing the device to a patient;

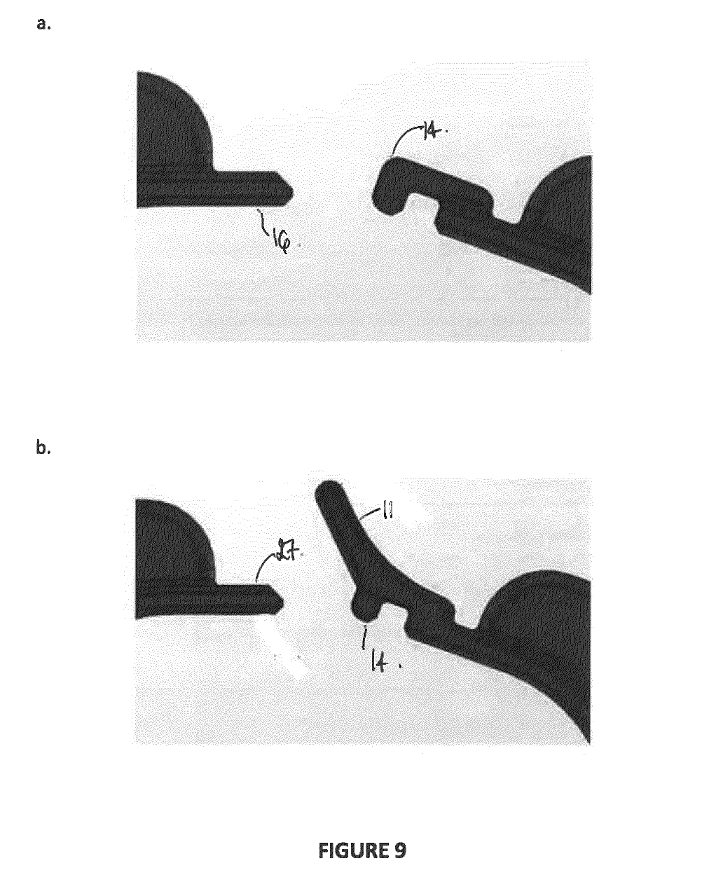

FIG. 9a is a perspective view of part of a fixation device according to one embodiment of the invention, where the fixation device is in an unlocked configuration;

FIG. 9b is a perspective view of part of a fixation device according to a second embodiment of the invention, wherein the fixation device is in an unlocked configuration;

FIG. 10 is a plan front view of a fixation device according to a second embodiment of the invention, where the fixation device is in a locked configuration;

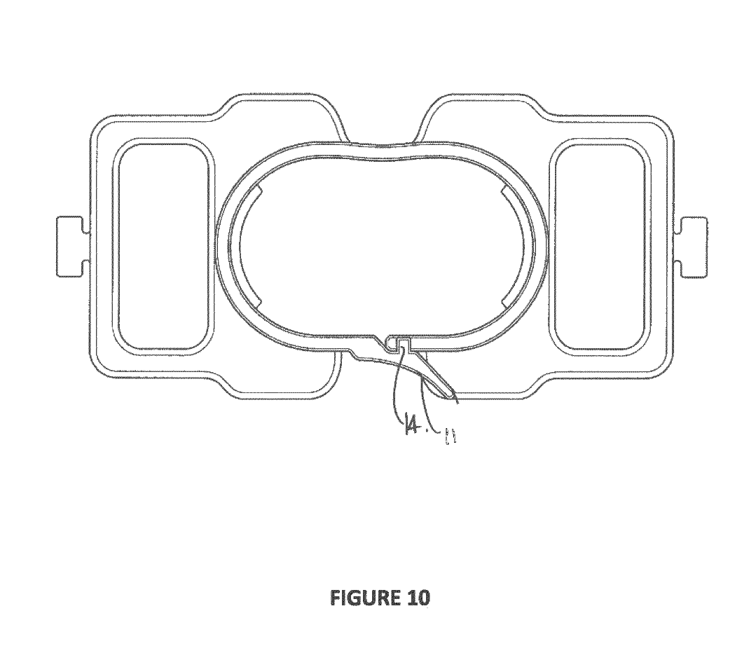

FIG. 11 is a perspective view of a fixation device according to a second embodiment of the invention, wherein the fixation device is in a locked configuration;

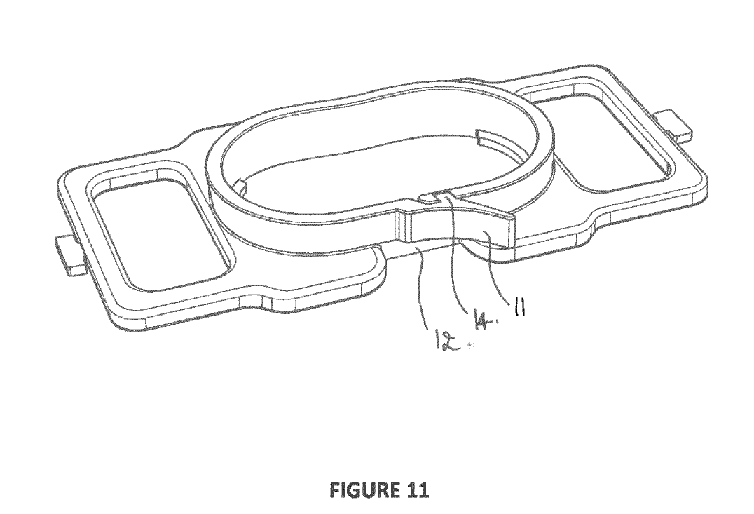

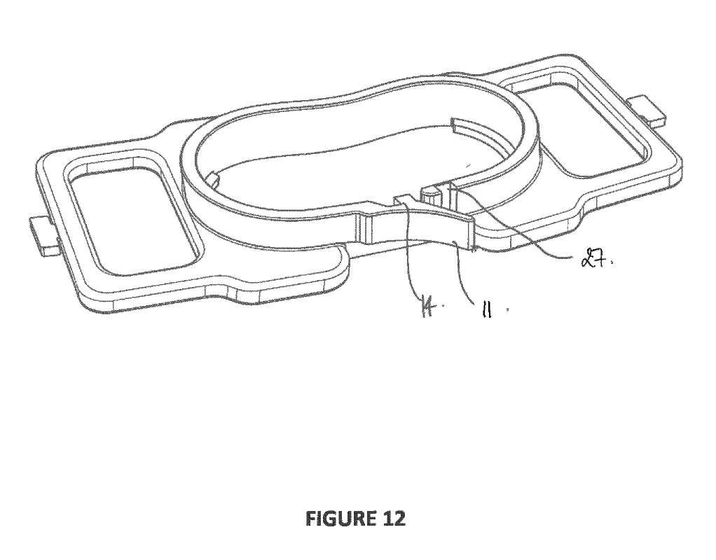

FIG. 12 is a perspective view of a fixation device according to a second embodiment of the invention, wherein the fixation device is in an unlocked configuration;

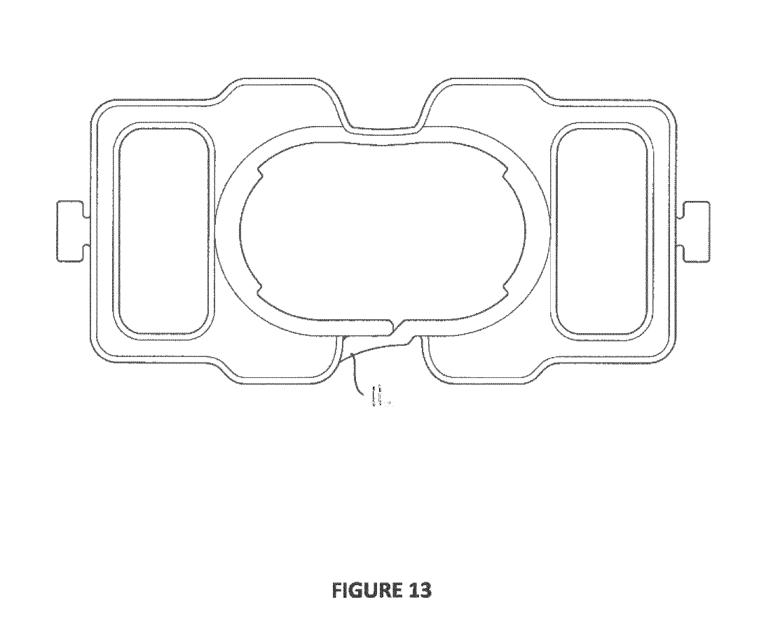

FIG. 13 is a plan rear view of a fixation device according to a second embodiment of the invention, where the fixation device is in a locked configuration;

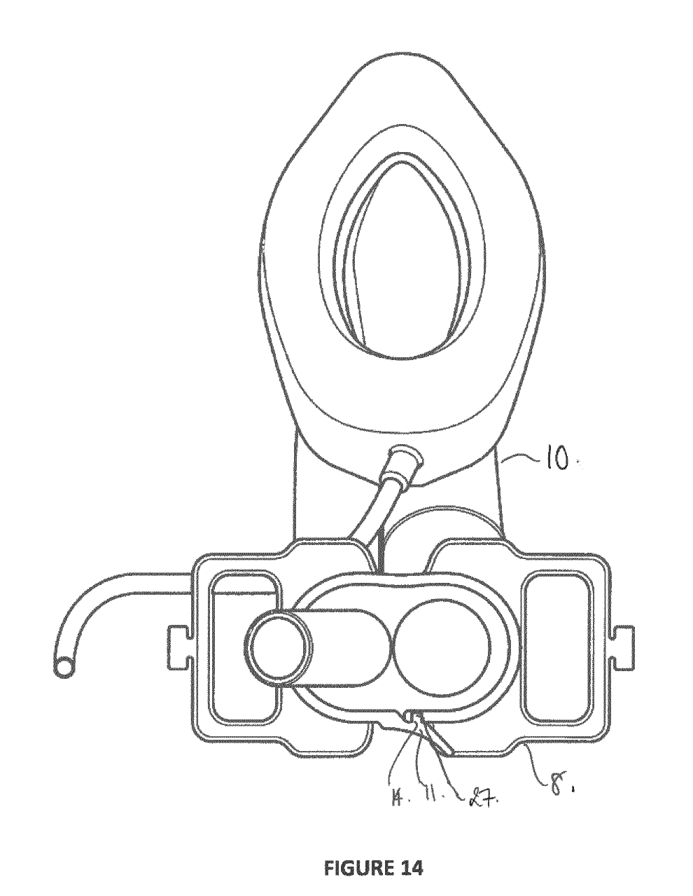

FIG. 14 is a front view of laryngeal mask airway device comprising a fixation device according to a second embodiment of the invention, where the fixation device is in a locked configuration;

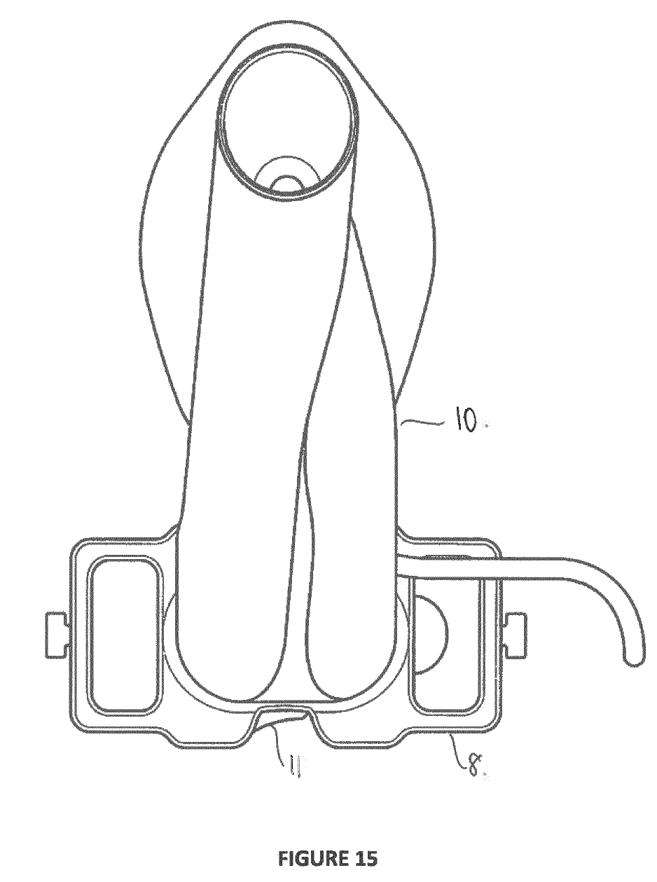

FIG. 15 is a rear view of laryngeal mask airway device comprising a fixation device according to a second embodiment of the invention, where the fixation device is in a locked configuration;

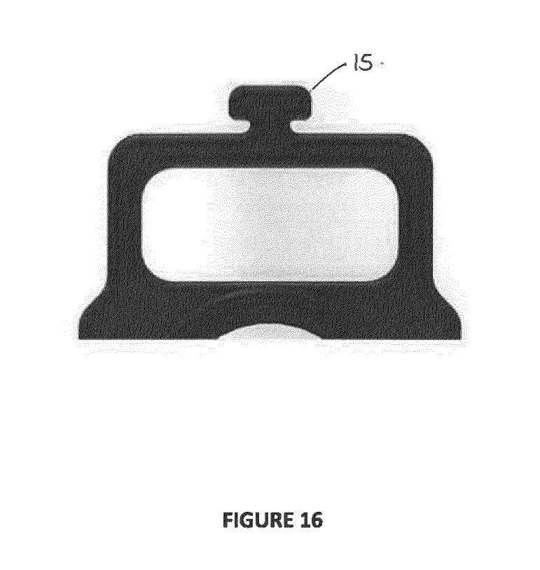

FIG. 16 is a plan view of a strap mount comprising a tab in accordance with an embodiment of the invention; and

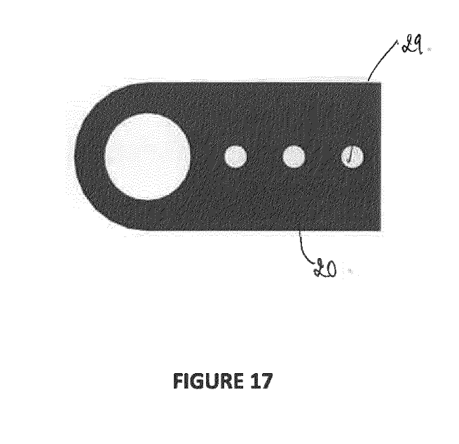

FIG. 17 is a plan view of part of a strap that may be used with the fixation device of the invention.

With reference to FIGS. 3, 4 and 5, there is provided a fixation device (8) for use with a laryngeal mask airway device (10), the fixation device (8) comprising releasable locking means (12) for releasably securing the fixation device to the laryngeal mask airway device.

Thus, the releasable locking means (12) is configured such that in a first configuration, the fixation device (8) may be secured to the laryngeal mask airway device (10) and locked in place, and in a second configuration, the fixation device (8) may be unlocked and removed from the laryngeal mask airway device (10). In the embodiment shown in FIG. 5, the fixation device (8) is secured to an outer surface of a biteblock (30) at the proximal end of the laryngeal mask airway device (10).

Preferably, the proximal end of the laryngeal mask airway device comprises an internal feature that strengthens this region of the device. Thus, securing the fixation device to the proximal end of the laryngeal mask airway device has the advantage of avoiding crushing of the laryngeal mask airway device. It is preferred that the internal strengthening means comprises part of the biteblock. In one embodiment, the internal strengthening means may comprise the junction between the gastro tube and airway tube of the device.

Thus, the fixation device (8) can be secured to the laryngeal mask airway device (10) (as shown, for example, in FIG. 5) and locked in place in a first configuration (as shown in FIGS. 3a, 3b and 3c) and/or unlocked and removed from the laryngeal mask airway device (10) in a second configuration (as shown in FIGS. 4a and 4b). The use of a fixation device (8) comprising releasable locking means (12) has the advantage that, in the locked position, it assists in holding the laryngeal mask airway device in the fully inserted configuration once the device has been inserted in a patient. Once the laryngeal mask airway device (10) has been removed from the patient's airway, the fixation device (8) may be unlocked and removed from the laryngeal mask airway device. The use of a fixation device therefore provides for secure attachment of the laryngeal mask airway device to a patient, and also advantageously allows the fixation device to be used with more than one laryngeal mask airway device, thus allowing for greater flexibility.

The releasable locking means (12) comprises male (14) and female (16) parts, wherein the male part is mutually engageable with the female part. The provision of such mutually engageable male and female parts assists in the locking and/or unlocking of the fixation device to facilitate attachment of the fixation device (8) to an outer surface of a laryngeal mask airway device (10) and/or removal of the fixation device (8) from the outer surface of the laryngeal mask airway device (10).

In the embodiment shown in FIGS. 3 and 4, the male and female parts comprise a hook (14) and aperture (16) combination, wherein the hook is releasably engageable with the aperture. Such arrangement facilitates the locking and/or unlocking the fixation device, thus assisting in securing the fixation device to a laryngeal mask airway device when the hook is engaged with the aperture and/or removing the fixation device from the laryngeal mask airway device when the hook and aperture combination are disengaged. In the embodiments shown in FIGS. 10 to 12, the male and female parts comprise a hook (14) and recess (27) combination, wherein the hook is releasably engageable with the recess, facilitating the locking and/or unlocking of the fixation device.

In the embodiment shown in FIGS. 10 to 13, the releasable locking means (12) comprises a lever (11) to facilitate the locking and/or unlocking of the first and second parts. Preferably, the lever is provided to facilitate locking and/or unlocking of the male (14) and female (16, 27) parts. Preferably, the lever extends from the first part, preferably the male part, of the releasable locking means, such that the user may use the lever to lock and/or unlock the fixation device. Preferably, the lever extends outwards from the releasable locking means in an arcuate shape. Advantageously, the arcuate shape allows the user to place their finger on the lever to facilitate the locking and/or unlocking of the first and second parts. Typically, when using the fixation device of the present invention, the user may need to reposition the fixation device with respect to the proximal end of the airway device, particularly in cases where the user may mistakenly position the fixation device in the wrong position with respect to the airway device, for example, where the user has a lack of experience in determining the optimal position of the fixation device. Thus, the provision of a lever advantageously assists the user in positioning and/or repositioning the first and second parts during locking and/or unlocking of the releasable locking means of the fixation device.

FIG. 9a shows the releasable locking means comprising a hook and aperture in accordance with a first embodiment of the invention, and FIG. 9b shows the releasable locking means comprising a hook and recess, and further comprising a lever in accordance with a second embodiment of the invention.

The fixation device (8) is shaped to correspond with the shape of the proximal end of the laryngeal mask airway device (10). As shown in FIG. 5, the inner surface of the fixation device corresponds to the shape of the outer surface of the proximal end of the laryngeal mask airway device. In this embodiment, the inner surface of the fixation device (8) corresponds to the shape of the outer surface of the biteblock (30) of the laryngeal mask airway device (10).

In one embodiment, the fixation device may comprise a ring shape. Preferably, the ring shape of the fixation device comprises a break, wherein the releasable locking means (12) is provided. As shown, for example, in FIG. 10, the lever (11) is positioned adjacent the break.

The releasable locking means (12) comprises first male (14) and second female parts (16), the first and second parts being mutually engageable. The provision of such mutually engageable parts assists in the locking and/or unlocking of the fixation device to a laryngeal mask airway device when in use.

As shown, for example, in FIG. 3b, the male and female parts comprise a hook (14) and aperture (16) combination, wherein the hook is releasably engageable with the aperture. The provision of a hook and aperture combination provides a means of easily locking and/or unlocking the fixation device, thus assisting in securing the fixation device to a laryngeal mask airway device (10) when the hook is engaged with the aperture, and subsequently removing the fixation device (8) from the laryngeal mask airway device (10) when the hook and aperture combination are disengaged.

With reference to FIG. 8, the fixation device (8) comprises a strap mount (18) around which a strap (20) can be removably fixed for securing the laryngeal mask airway device (10) to a patient. The provision of a fixation device (8) comprising a strap mount (18) assists in more reliably securing the laryngeal mask airway device (10) in the fully inserted configuration once it has been inserted in a patient. It is preferred that the strap has a length of around 650 mm. The provision of a strap having a length of around 650 mm overcomes the problems associated with straps of shorter lengths where in some cases they did not fit all sizes of patient.

The strap mount (18) comprises at least one mount bar (22). With reference, for example, to FIGS. 3, 4 and 8, the strap mount (18) comprises two mount bars (22). The provision of a strap mount having one or two bars assists in attaching one or more straps to the strap mount, to assist in securing the laryngeal mask airway device (10) to a patient.

The at least one mount bar (22) is disposed between two arms (24) which extend in a direction substantially perpendicular to the longitudinal axis of the laryngeal mask airway device (10), wherein a mount bar (22) is disposed on either side of the laryngeal mask airway device (10). Thus, the laryngeal mask airway device (10) is more reliably held in position when inserted within a patient. The mount bars (22) are provided at the proximal end of the laryngeal mask airway device (10).

As shown in FIGS. 3 and 4, the fixation device (8) comprises two tabs (15), each tab being provided on a mount bar. Each tab (15) is shaped to correspond with an aperture (17) provided on a strap (as shown in FIG. 8), such that the at least one tab is provided to hold the strap in place when the device is inserted within a patient.

Preferably, the at least one tab (15) has dimensions that allow it to be robust, preventing breakage of the at least one tab and facilitating attachment of the strap to the at least one tab. The provision of a tab (15) of larger dimensions is shown in FIGS. 10 to 16.

With reference to FIG. 17, the strap comprises a plurality of holes (29) for attachment to the at least one tab (15) and/or the strap mount (18). Preferably, the strap comprises around 20 holes. Preferably, the diameter of the holes is sized to correspond with the size of the at least one tab. Preferably, the diameter of the holes is around 4 mm.

The fixation device (8) comprises a plastics material. In one embodiment, the fixation device may comprise polyvinyl chloride or silicone.

With reference to FIGS. 3 to 8, the fixation device (8) further comprises means for repositioning (26, 28) the fixation device with respect to the laryngeal mask airway device (10) at one or more different positions. Thus, the fixation device (8) can be secured in a plurality of positions with respect to the outer surface of the laryngeal mask airway device (10), thus allowing the laryngeal mask airway device to be adapted for patients having different sizes of airways.

The means for repositioning (26, 28) comprises a male part (26) provided on the fixation device (8) which is mutually engageable with a female part (28) provided on an outer surface of the laryngeal mask airway device (10). In another embodiment, the means for repositioning (26, 28) may comprise a female part provided on the fixation device which is mutually engageable with a male part provided on an outer surface of the laryngeal mask airway device (10). Thus, the fixation device (8) may be releasably secured at different positions on the outer surface of the laryngeal mask airway device.

In the embodiment shown in FIGS. 3 and 4, the means for repositioning comprises two ridges (26) provided on an inner surface of the fixation device, wherein the two ridges (26) engage with a corresponding recess (28) provided on an outer surface of the laryngeal mask airway device (10).

As shown, for example, in FIG. 5, the at least one recess (28) is provided on an outer surface of a biteblock (30) of the laryngeal mask airway device (4).

The at least one ridge (26) is mutually engageable with the at least one recess (28). Thus, the fixation device (8) may be releasably secured at different positions of the outer surface of the laryngeal mask airway device (10).

The at least one recess (28) extends along a side of the biteblock (30) in a direction substantially perpendicular to the longitudinal axis of the laryngeal mask airway device (10).

The fixation device (8) is movable between a first position and a second position along the longitudinal length of the laryngeal mask airway device and releasably securable thereto.

With reference to FIGS. 3 to 8, there is also provided a fixation device (8) for use with a laryngeal mask airway device (10), the fixation device (8) comprising releasable repositioning means (26, 28) for releasably securing the fixation device (8) to the laryngeal mask airway device (10) at a plurality of different positions. Thus, the fixation device (8) can be secured in a plurality of positions with respect to the outer surface of the laryngeal mask airway device (10), thus allowing the device to be adapted for patients having different sizes of airways.

The means for repositioning comprises a male part (26) provided on the fixation device (8) which is mutually engageable with a female part (28) provided on an outer surface of the laryngeal mask airway device (10). Thus, the fixation device (8) may be releasably secured at different positions on the outer surface of the laryngeal mask airway device (10).

In the embodiment shown in FIGS. 3 and 4, the means for repositioning comprises two ridges (26) provided on an inner surface of the fixation device (8), wherein the ridges (26) engage with a corresponding recess (28) provided on an outer surface of the laryngeal mask airway device.

In the embodiment shown in FIG. 5, the at least one recess (28) is provided on an outer surface of a biteblock (30) of the device (10).

Thus, the at least one ridge (26) is mutually engageable with the at least one recess (28) such that the fixation device (8) may be releasably secured at different positions of the outer surface of the laryngeal mask airway device (10).

The at least one recess (26) extends along a side of the biteblock (30) in a direction substantially perpendicular to the longitudinal axis of the laryngeal mask airway device (10).

Advantageously, the fixation device (8) is movable between a first and second position along the longitudinal length of the laryngeal mask airway device (10) and releasably securable thereto.

The fixation device (8) further comprises releasable locking means (12) comprising first (14) and second (16) parts, the first and second parts being mutually engageable.

In the locked configuration, the releasable locking means assists in holding the laryngeal mask airway device (10) in the fully inserted configuration once the device (10) has been inserted in a patient. Once the laryngeal mask airway device (10) has been removed from the patient's airway, the fixation device (8) may be unlocked and removed from the laryngeal mask airway device. The use of a fixation device having releasable locking means allows the fixation device to be used with more than one laryngeal mask airway device, thus allowing for greater flexibility.

In the embodiment shown in FIGS. 3 and 4, the first and second parts comprise a hook (14) and aperture (16) combination, wherein the hook is releasably engageable with the aperture. Such arrangement provides a means of easily locking and/or unlocking the fixation device, thus assisting in securing the fixation device to a laryngeal mask airway device when the hook is engaged with the aperture and/or removing the fixation device (8) from the laryngeal mask airway device (10) when the hook and aperture combination are disengaged. In the embodiments shown in FIGS. 10 to 12, the male and female parts comprise a hook (14) and recess (27) combination, wherein the hook is releasably engageable with the recess.

In the embodiment shown in FIGS. 10 to 13, the releasable locking means (12) comprises a lever (11) to facilitate the locking and/or unlocking of the first and second parts. Preferably, the lever is provided to facilitate locking and/or unlocking of the male (14) and female (16, 27) parts. Preferably, the lever extends from the first part, preferably the male part, such that the user may use the lever to lock and/or unlock the fixation device. Preferably, the lever extends outwards from the releasable locking means in an arcuate shape. Advantageously, the arcuate shape allows the user to place their finger on the lever to facilitate the locking and/or unlocking of the first and second parts. Typically, when using the fixation device of the present invention, the user may need to reposition the fixation device with respect to the proximal end of the airway device, particularly in cases where the user may mistakenly position the fixation device in the wrong position with respect to the airway device, for example, where the user has a lack of experience in determining the optimal position of the fixation device. Thus, the provision of a lever advantageously assists the user in positioning and/or repositioning the first and second parts during locking and/or unlocking of the releasable locking means of the fixation device.

With reference to FIG. 5, there also is provided a laryngeal mask airway device (10), comprising at least one airway tube (32) and a mask (34) carried at one end of the at least one airway tube (32), the mask (34) having a distal end (36) and a proximal end (38) and a peripheral formation (40) capable of conforming to, and of fitting within, the actual and potential space behind the larynx of the patient so as to form a seal around the circumference of the laryngeal inlet, the peripheral formation (40) surrounding a hollow interior space or lumen of the mask and the at least one airway tube (32) opening into the lumen of the mask, the peripheral formation (40) establishing separation between a laryngeal chamber side and a pharyngeal side, the device further comprising a fixation device comprising releasable locking means (12) for releasably securing the fixation device to the laryngeal mask airway device. Thus, the laryngeal mask airway device is held in the fully inserted configuration once the device (10) has been inserted in a patient. Once the laryngeal mask airway device has been removed from the patient's airway, the fixation device may be unlocked and removed from the laryngeal mask airway device. The fixation device may advantageously be used with more than one laryngeal mask airway device, thus allowing for greater flexibility.

With reference to FIG. 5, there is also provided there is provided a laryngeal mask airway device (10), comprising at least one airway tube (32) and a mask (34) carried at one end of the at least one airway tube, the mask (34) having a distal end (36) and a proximal end (38) and a peripheral formation (40) capable of conforming to, and of fitting within, the actual and potential space behind the larynx of the patient so as to form a seal around the circumference of the laryngeal inlet, the peripheral formation (40) surrounding a hollow interior space or lumen of the mask and the at least one airway tube (32) opening into the lumen of the mask, the peripheral formation (40) establishing separation between a laryngeal chamber side and a pharyngeal side, the device further comprising a fixation device comprising releasable repositioning means (18) for releasably securing the fixation device to the laryngeal mask airway device at a plurality of different positions. Such arrangement advantageously allows the device to be adapted for patients having different sizes of airways.

The laryngeal mask airway device (10) further comprises a conduit (42) adapted for passage of an endoscope into the oesophagus of a patient when the mask is in place, the conduit (42) including a distal end for cooperation with the oesophageal sphincter of the patient, the conduit having a laryngeal side, a pharyngeal side, and with respect thereto, a right side and a left side, and an outlet (44). Thus, the larungeal mask airway device of the present invention has the combined benefits of establishing an airway within a patient and protecting the airway from regurgitation or vomiting that might be caused by endoscopy by providing a close cooperation between the oesophageal sphincter of the patient and the distal end of the conduit. Thus, the device of the present invention advantageously assists in the safe and accurate insertion of an endoscope within the oesophagus of a patient, whilst at the same time establishing an airway within the patient.

Preferably, at the outlet (44) the width between the left side and the right side is smaller than the width of the conduit (42).

Preferably, at the outlet (44), the pharyngeal side is terminated proximally of the laryngeal side.

Preferably, at the outlet (44), the width of the conduit wall is reduced. Typically, the width reduction is at the left and the right side of the outlet.

It is preferred that the distal end of the conduit (42) is provided at an angle of about 30 degrees, relative to the longitudinal axis of the conduit.

Preferably, the conduit (42) includes an internal sleeve (45) at its proximal end.

Preferably, the laryngeal mask airway device further comprises a connector (46). Typically, the sleeve (45) is integrally formed with the connector (46).

Preferably, the conduit (42) has a large bore diameter. Typically, the conduit has a diameter of between about 5 and 25 mm, more typically between about 10 and 20 mm, and most typically about 15 mm, depending on the size of the laryngeal mask airway device. The diameter of the conduit advantageously allows the passage of an endoscope through the conduit. Thus, the conduit typically forms an “operating channel” through which a surgeon can view the upper gastrointestinal tract of a patient. In addition, the diameter of the conduit may be varied depending on the anatomy of a patient. Preferably, the radial wall thickness of the conduit is between about 1 to 2 mm.

Typically, the laryngeal mask airway device further comprises an inflation line (19) preferably comprising a pressure valve (21).

Typically, the laryngeal mask airway device of the present invention is inserted into the upper oesophageal sphincter by means of the tip of the cuff, which provides guided insertion of the device. Furthermore, the distal flexibility of the tip of the device may assist in tracking the posterior curvature of the throat of a patient upon insertion of the device and reduces trauma to the throat of the patient.

The use of a laryngeal mask airway device in accordance with the present invention in combination with an endoscope is safer than use of the endoscope alone and advantageously allows the endoscope to be inserted within the oesophagus of a patient for a longer period of time.

Typically, the peripheral formation may be inflatable. Preferably, the peripheral formation comprises an inflatable cuff. Typically, the cuff is capable of conforming to and fitting within the actual and potential space behind the larynx of a patient so as to form a seal around the laryngeal inlet. Typically, the cuff extends from a proximal end to a distal end.

With reference to the drawings, there is provided a kit comprising a laryngeal mask airway device and a fixation device according to the first and/or second aspects of the invention.

With reference to the drawings, there is provided a method of releasably securing the fixation device (8) of the first aspect to a laryngeal mask airway device (10). In use, the fixation device (8) of the first aspect is placed around the proximal end of a laryngeal mask airway device (10). The hook (14) of the releasable locking means engages the aperture (16) or recess (27) and the fixation device is locked in place. A lever (11) may be used to facilitate the unlocking of the hook from the aperture or recess such that the user may unlock the releasable locking means and remove the fixation device from the laryngeal mask.

With reference to the drawings, there is provided a method of repositioning the fixation device of the second aspect to a laryngeal mask airway device at a plurality of different positions. In use, the fixation device (8) of the second aspect is placed around the proximal end of a laryngeal mask airway device (10). The fixation device comprises means for repositioning (26, 28) which allow the fixation device to be secured in a plurality of positions with respect to the outer surface of the laryngeal mask airway device.

With reference to the drawings, there is provided a method of making a laryngeal mask airway device according to the third and/or fourth aspect, comprising the steps of forming a laryngeal mask airway device, forming a fixation device according to the first and/or second aspect separately from the laryngeal mask airway device, wherein the fixation device is adapted to be releasably securable to the laryngeal mask airway device.

With reference to the drawings, there is provided a method of inserting a laryngeal mask airway device into a patient, the method comprising the steps of inserting the mask to the required position and securing the mask in the required position by adjusting the position of the releasable fixation device of the first and/or second aspect with respect to the proximal end of the laryngeal mask airway device.

1. A fixation device for use with a laryngeal mask airway device, the fixation device comprising releasable locking means for releasably securing the fixation device to the laryngeal mask airway device.

2. The fixation device of claim 1, wherein the releasable locking means is configured such that in a first configuration, the fixation device may be secured to the laryngeal mask airway device and locked in place, and in a second configuration, the fixation device may be unlocked and removed from the laryngeal mask airway device.

3. The fixation device of claim 1 or 2, wherein the releasable locking means comprises first and second parts, the first and second parts being mutually engageable.

4. The fixation device of claim 1, 2 or 3, the releasable locking means comprising male and female parts, wherein the male part is mutually engageable with the female part.

5. The fixation device of claim 4, wherein the male and female parts comprise a hook and aperture combination, wherein the hook is releasably engageable with the aperture.

6. The fixation device of claim 4, wherein the male and female parts comprise a hook and recess combination, wherein the hook is releasably engageable with the recess.

7. The fixation device of any preceding claim, wherein the releasable locking means is biased towards the locked configuration.

8. The fixation device of any preceding claim, wherein the releasable locking means further comprises a lever to facilitate locking and/or unlocking of the fixation device.

9. The fixation device of any preceding claim, wherein the fixation device is shaped to correspond with the shape of the proximal end of the laryngeal mask airway device.

10. The fixation device of any preceding claim, wherein the inner surface of the fixation device corresponds to the shape of the outer surface of the proximal end of the laryngeal mask airway device.

11. The fixation device of any preceding claim, wherein the fixation device is shaped to correspond with the shape of a biteblock of the laryngeal mask airway device.

12. The fixation device of any preceding claim, wherein the inner surface of the fixation device corresponds to the shape of the outer surface of the biteblock of the laryngeal mask airway device.

13. The fixation device of any preceding claim, wherein the fixation device is secured to an outer surface of the laryngeal mask airway device.

14. The fixation device of any preceding claim, wherein the fixation device is secured to an outer surface of a biteblock of the laryngeal mask airway device.

15. The fixation device of any preceding claim, wherein the fixation device comprises a ring-like shape.

16. The fixation device of any preceding claim, comprising a strap mount around which a strap can be releasably fixed for securing the laryngeal mask airway device to a patient.

17. The fixation device of claim 16, comprising at least one mount bar.

18. The fixation device of claim 17, wherein the at least one mount bar is disposed between two arms which extend in a direction substantially perpendicular to the longitudinal axis of the device, wherein a mount bar is disposed on either side of the device.

19. The fixation device of any preceding claim, further comprising at least one tab.

20. The fixation device of claim 19, comprising two tabs.

21. The fixation device of claim 20, wherein each tab is provided on a mount bar.

22. The fixation device of claim 19, 20 or 21, wherein the at least one tab is shaped to correspond with an aperture provided on a strap, such that the at least one tab is provided to hold the strap in place when the device is inserted within a patient.

23. The fixation device of any preceding claim, comprising a plastics material.

24. The fixation device of any preceding claim, further comprising means for repositioning the fixation device with respect to the laryngeal mask airway device at one or more different positions.

25. The fixation device of claim 24, wherein the means for repositioning comprises mutually interengageable male and female parts.

26. The fixation device of claim 24 or 25, wherein the means for repositioning comprises at least one ridge provided on an inner surface of the fixation device.

27. The fixation device of claim 26, comprising two ridges.

28. The fixation device of any one of claims 24 to 27, wherein the at least one ridge engages with at least one recess provided on an outer surface of the laryngeal mask airway device.

29. The fixation device of claim 28, wherein the at least one recess is provided on an outer surface of a biteblock of the laryngeal mask airway device.

30. The fixation device of claim 28 or 29, wherein the at least one ridge is mutually engageable with the at least one recess.

31. The fixation device of claim 29 or 30, wherein the at least one recess extends along a side of the bite block in a direction substantially perpendicular to the longitudinal axis of the laryngeal mask airway device.

32. The fixation device of any one of claims 24 to 31, wherein the fixation device is movable between a first position and a second position along the longitudinal length of the laryngeal mask airway device and releasably securable thereto.

33. A fixation device for use with a laryngeal mask airway device, the fixation device comprising releasable repositioning means for releasably securing the fixation device to the laryngeal mask airway device at a plurality of different positions.

34. The fixation device of claim 33, wherein the means for repositioning comprises mutually interngageable male and female parts.

35. The fixation device of claim 33 or 34, the means for repositioning comprising at least one ridge provided on an inner surface of the fixation device.

36. The fixation device of claim 35, comprising two ridges.

37. The fixation device of any one of claims 33 to 36, wherein the at least one ridge engages with at least one recess provided on an outer surface of the laryngeal mask airway device.

38. The fixation device of claim 37, wherein the at least one recess is provided on an outer surface of a biteblock of the device.

39. The fixation device of claim 38, wherein the at least one ridge is mutually engageable with the at least one recess.

40. The fixation device of any one of claims 37 to 39, wherein the at least one recess extends along a side of the biteblock in a direction substantially perpendicular to the longitudinal axis of the laryngeal mask airway device.

41. The fixation device of any one of claims 33 to 40, wherein the fixation device is movable to between a first and second position along the longitudinal length of the laryngeal mask airway device and releasably securable thereto.

42. The fixation device of any one of claims 33 to 41, further comprising releasable locking means for releasably securing the fixation device to the laryngeal mask airway device.

43. The fixation device of claim 42, wherein the releasable locking means is configured such that in a first configuration, the fixation device may be secured to the laryngeal mask airway device and locked in place, and in a second configuration, the fixation device may be unlocked and removed from the laryngeal mask airway device.

44. The fixation device of claim 42 or 43, wherein the releasable locking means comprises first and second parts, the first and second parts being mutually engageable.

45. The fixation device of claim 42, 43 or 44, wherein the releasable locking means comprises male and female parts, wherein the male part is mutually engageable with the female part.

46. The fixation device of claim 45, wherein the male and female parts comprise a hook and aperture combination, wherein the hook is releasably engageable with the aperture.

47. The fixation device of claim 45, wherein the male and female parts comprise a hook and recess combination, wherein the hook is releasably engageable with the recess.

48. The fixation device of any one of claims 42 to 47, wherein the releasable locking means is biased towards the locked configuration.

49. The fixation device of any one of claims 42 to 48, wherein the releasable locking means further comprises a lever to facilitate locking and/or unlocking of the fixation device.

50. The fixation device of any one of claims 33 to 49, wherein the fixation device is shaped to correspond with the shape of the proximal end of the laryngeal mask airway device.

51. The fixation device of any one of claims 33 to 50, wherein the inner surface of the fixation device corresponds to the shape of the outer surface of the proximal end of the laryngeal mask airway device.

52. The fixation device of any one of claims 33 to 51, wherein the fixation device is shaped to correspond with the shape of a biteblock of the laryngeal mask airway device.

53. The fixation device of any one of claims 33 to 52, wherein the inner surface of the fixation device corresponds to the shape of the outer surface of the biteblock of the laryngeal mask airway device.

54. The fixation device of any one of claims 33 to 53, wherein the fixation device is secured to an outer surface of the laryngeal mask airway device.

55. The fixation device of any one of claims 33 to 54, wherein the fixation device is secured to an outer surface of a biteblock of the laryngeal mask airway device.

56. The fixation device of any one of claims 42 to 55, wherein the fixation device comprises a ring-like shape.

57. The fixation device of any one of claims 33 to 56, comprising a strap mount around which a strap can be removably fixed for securing the laryngeal mask airway device to a patient.

58. The fixation device of claim 57, comprising at least one mount bar.

59. The fixation device of claim 58, wherein the at least one mount bar is disposed between two arms which extend in a direction substantially perpendicular to the longitudinal axis of the device, wherein a mount bar is disposed on either side of the device.

60. The fixation device of any one of claims 33 to 59, further comprising at least one tab.

61. The fixation device of claim 60, comprising two tabs.

62. The fixation device of claim 61, wherein each tab is provided on a mount bar.

63. The fixation device of claim 58, 59 or 60, wherein the at least one tab is shaped to correspond with an aperture provided on a strap, such that the at least one tab is provided to hold the strap in place when the device is inserted within a patient.

64. The fixation device of any one of claims 33 to 63, comprising a plastics material.

65. A laryngeal mask airway device, comprising at least one airway tube and a mask carried at one end of the at least one airway tube, the mask having a distal end and a proximal end and a peripheral formation capable of conforming to, and of fitting within, the actual and potential space behind the larynx of the patient so as to form a seal around the circumference of the laryngeal inlet, the peripheral formation surrounding a hollow interior space or lumen of the mask and the at least one airway tube opening into the lumen of the mask, the peripheral formation establishing separation between a laryngeal chamber side and a pharyngeal side, the device further comprising a fixation device comprising releasable locking means for releasably securing the fixation device to the laryngeal mask airway device.

66. The laryngeal mask airway device of claim 65, wherein the releasable locking means is configured such that in a first configuration, the fixation device may be secured to the laryngeal mask airway device and locked in place, and in a second configuration, the fixation device may be unlocked and removed from the laryngeal mask airway device.

67. The laryngeal mask airway device of claim 65 or 66, wherein the releasable locking means comprises first and second parts, the first and second parts being mutually engageable.

68. The laryngeal mask airway device of claim 65, 66 or 67, the releasable locking means comprises male and female parts, wherein the male part is mutually engageable with the female part.

69. The laryngeal mask airway device of claim 68, wherein the male and female parts comprise a book and aperture combination, wherein the hook is releasably engageable with the aperture.

70. The fixation device of claim 68, wherein the male and female parts comprise a hook and recess combination, wherein the hook is releasably engageable with the recess.

71. The fixation device of any one of claim 65 to 70, wherein the releasable locking means is biased towards the locked configuration.

72. The fixation device of any claims 65 to 71, wherein the releasably locking means further comprises a lever to facilitate locking and/or unlocking of the fixation device.

73. The laryngeal mask airway device of any one of claims 65 to 72, wherein the fixation device is shaped to correspond with the shape of the proximal end of the laryngeal mask airway device.

74. The laryngeal mask airway device of any one of claims 65 to 73, wherein the inner surface of the fixation device corresponds to the shape of the outer surface of the proximal end of the laryngeal mask airway device.

75. The laryngeal mask airway device of any one of claims 65 to 74, wherein the fixation device is shaped to correspond with the shape of a biteblock of the laryngeal mask airway device.

76. The laryngeal mask airway device of any one of claims 65 to 75, wherein the inner surface of the fixation device corresponds to the shape of the outer surface of the biteblock of the laryngeal mask airway device.

77. The laryngeal mask airway device of any one of claims 65 to 76, wherein the fixation device is secured to an outer surface of the laryngeal mask airway device.

78. The laryngeal mask airway device of any one of claims 65 to 77, wherein the fixation device is secured to an outer surface of a biteblock of the laryngeal mask airway device.

79. The laryngeal mask airway device of any one of claims 65 to 78, wherein the fixation device comprises a ring-like shape.

80. The laryngeal mask airway device of any one of claims 65 to 79, comprising a strap mount around which a strap can be removably fixed for securing the laryngeal mask airway device to a patient.

81. The laryngeal mask airway device of claim 80, comprising at least one mount bar.

82. The laryngeal mask airway device of claim 81, wherein the at least one mount bar is disposed between two arms which extend in a direction substantially perpendicular to the longitudinal axis of the device, wherein a mount bar is disposed on either side of the device.

83. The laryngeal mask airway device of any one of claims 65 to 82, further comprising at least one tab.

84. The laryngeal mask airway device of claim 83, comprising two tabs.

85. The laryngeal mask airway device of claim 84, wherein each tab is provided on a mount bar.

86. The laryngeal mask airway device of claim 83, 84 or 85, wherein the at least one tab is shaped to correspond with an aperture provided on a strap, such that the at least one tab is provided to hold the strap in place when the device is inserted within a patient.

87. The laryngeal mask airway device of any one of claims 65 to 86, comprising a plastics material.

88. The laryngeal mask airway device of any one of claims 65 to 87, further comprising means for repositioning the fixation device with respect to the laryngeal mask airway device at one or more different positions.

89. The laryngeal mask airway device of claim 88, wherein the means for repositioning comprises mutually interengageable male and female parts.

90. The laryngeal mask airway device of claim 88 or 89, wherein the means for repositioning comprises at least one ridge provided on an inner surface of the fixation device.

91. The laryngeal mask airway device of claim 90, comprising two ridges.

92. The laryngeal mask airway device of any one of claim 90 or 91, wherein the at least one ridge engages with at least one recess provided on an outer surface of the laryngeal mask airway device.

93. The laryngeal mask airway device of any one of claims 65 to 92, further comprising a biteblock.

94. The laryngeal mask airway device of claim 93, wherein the at least one recess is provided on an outer surface of the biteblock.

95. The laryngeal mask airway device of any one of claims 90 to 94, wherein the at least one ridge is mutually engageable with the at least one recess.

96. The laryngeal mask airway device of any one of claims 92 to 95 wherein the at least one recess extends along a side of the biteblock in a direction substantially perpendicular to the longitudinal axis of the laryngeal mask airway device.

97. The laryngeal mask airway device of any one of claims 92 to 96, wherein the fixation device is movable between a first position and a second position along the longitudinal length of the laryngeal mask airway device and releasably securable thereto.

98. The laryngeal mask airway device of any one of claims 92 to 97, further comprising a conduit adapted for passage of an endoscope into the oesophagus of a patient when the mask is in place, the conduit including a distal end for cooperation with the oesophageal sphincter of the patient, the conduit having a laryngeal side, a pharyngeal side, and with respect thereto, a right side and a left side, and an outlet.

99. The laryngeal mask airway device of claim 98, wherein at the outlet the width between the left side and the right side is smaller than the width of the conduit.

100. The laryngeal mask airway device of claim 98 or 99, wherein at the outlet, the pharyngeal side is terminated proximally of the laryngeal side.

101. The laryngeal mask airway device of any one of claims 98 to 100, wherein at the outlet, the width of the conduit wall is reduced.

102. The laryngeal mask airway device of claim 101, wherein the width reduction is at the left and the right side of the outlet.

103. The laryngeal mask airway device of any one of claims 98 to 102, wherein the distal end of the conduit is provided at an angle of about 30 degrees, relative to the longitudinal axis of the conduit.

104. The laryngeal mask airway device of any one of claims 98 to 103, wherein the conduit includes an internal sleeve at its proximal end.

105. The laryngeal mask airway device of any one of claims 65 to 104, further comprising a connector.

106. The laryngeal mask airway device of claim 104 or 105, wherein the sleeve is integrally formed with the connector.

107. A laryngeal mask airway device, comprising at least one airway tube and a mask carried at one end of the at least one airway tube, the mask having a distal end and a proximal end and a peripheral formation capable of conforming to, and of fitting within, the actual and potential space behind the larynx of the patient so as to form a seal around the circumference of the laryngeal inlet, the peripheral formation surrounding a hollow interior space or lumen of the mask and the at least one airway tube opening into the lumen of the mask, the peripheral formation establishing separation between a laryngeal chamber side and a pharyngeal side, the device further comprising a fixation device comprising repositioning means for releasably securing the fixation device to the laryngeal mask airway device at a plurality of different positions.

108. A kit comprising a laryngeal mask airway device and a fixation device according to any one of claims 1 to 64.

109. A method of releasably securing the fixation device of any one of claims 1 to 32 to a laryngeal mask airway device.

110. A method of repositioning the fixation device of any one of claims 33 to 64 with respect to a laryngeal mask airway device at a plurality of different positions.

111. A method of making a laryngeal mask airway device according to any one of claims 65 to 107, comprising the steps of forming a laryngeal mask airway device, forming a fixation device according to any one of claim 1 to 64 separately from the laryngeal mask airway device, wherein the fixation device is adapted to be releasably securable to the laryngeal mask airway device.

112. A method of inserting a laryngeal mask airway device into a patient, the method comprising the steps of inserting the mask to the required position and securing the mask in the required position by adjusting the position of the releasable fixation device according to any one of claims 1 to 64, with respect to the proximal end of the laryngeal mask airway device.

113. A fixation device substantially as hereinbefore described with reference to the accompanying drawings.

114. A laryngeal mask airway device substantially as hereinbefore described with reference to the accompanying drawings.
